(19) United States
(12) Patent Application Publication
(10) Pub. No.: US 2025/0302792 A1
(43) Pub. Date: Oct. 2, 2025

(54) ACTIVE SUBSTANCES FOR MEDICAL USE

(71) Applicant: inflamed pharma GmbH, Jena (DE)

(72) Inventors: Christina Ehrhardt, Jena (DE); Sarah Koenig, Grossbartloff (DE); Josefine Schroeder, Jena (DE)

(21) Appl. No.: 18/864,400

(22) PCT Filed: May 10, 2023
(86) PCT No.: PCT/EP2023/062420

§ 371 Date: Nov. 8, 2024

(30) Foreign Application Priority Data
May 10, 2022 (EP) 22172462.8

(51) Int. Cl. A61K 31/245; A61K 31/506

(52) U.S. Cl. CPC A61K 31/245; A61K 31/506

(57) ABSTRACT

The present invention relates to an amine (AM) according to general formula (I); a carbonic acid adduct (KA) and a pharmaceutical composition (PZ) for use in the treatment of Aspergillus fumigatus infections and superinfections with influenza viruses and Aspergillus spp.

### TECHNICAL FIELD

The present invention relates to an amine (AM) of the general formula (I), to a carbonic acid adduct (KA) and to a pharmaceutical composition (PZ) for use in treatment of infections with Aspergillus fumigatus and of superinfections with influenza viruses and Aspergillus spp.

### TECHNICAL BACKGROUND

Influenza viruses are continuing to cause severe respiratory pathway disorders that contribute to considerable morbidity and mortality. Seasonal epidemics are responsible for 3-5 million severe cases and an estimated 300 000-500 000 deaths per year globally (1).

Furthermore, influenza A viruses harbor the potential to cause pandemics, which caused several million deaths in the past, as evidenced, for example, by the outbreak of Spanish flu in 1918/19(2). While most people recover from an infection, others experience complications such as pulmonary inflammation, which is caused either by the virus itself or additional pathogens, including bacteria, among which Streptococcus pneumoniae, Staphylococcus aureus and Haemophilus influenzae play a major role (3). In the case of superinfections with influenza viruses and bacteria, the reasons for severe progressions have been well examined, and novel treatment options have been the focus of interest. However, this is not the case for superinfections with influenza viruses and fungi (4, 5).

Nevertheless, it has become clear that infections by fungi such as Aspergillus spp., especially Aspergillus fumigatus, occur in patients infected with influenza virus and cause elevated morbidity and mortality (5-9). A common problem with secondary infections with fungi is that they are often recognized too late (10, 11). As well as elevated pathogenicity in the case of superinfections with influenza viruses and Aspergillus fumigatus, the limited availability of potent anti-infectives against the various pathogens is of major importance (5, 6, 12). The high variability of the influenza viruses and the constant occurrence of new strains, and the rapid development of resistance by influenza viruses against the available medicaments (13-16) in combination with the late recognition of fungal infections (10, 11), are the main reasons for the poor treatment options. Treatment of superinfections by influenza viruses and fungi with a single active ingredient has not yet been possible to date.

Even though intensive studies and enormous advances in possible treatments have taken place in the past century, influenza viruses still constitute a serious threat to the population. Seasonal epidemics are attributable to the continuous point mutations in the genome of influenza A and B viruses (antigenic drift), which lead to a change in protein structures. Furthermore, influenza A viruses have the potential to cause pandemic outbreaks (2, 20-22). In the case of infection with more than one virus subtype per individual, exchange of gene segments (antigen shifting) can lead to virus subtypes that contain the properties of the parent strains in a new combination (20, 21). As a result, the immune system of infected hosts is naive and incapable of efficiently combating the new pathogens. There were several pandemics in the last century, among which the “Spanish flu” of 1918/19 had the most serious consequences (23). The influenza virus strain responsible for the pandemic outbreak of 2009 is still circulating today in the population together with the H3N2 line from 1968 and two different lines of the influenza B virus (20).

While viral and bacterial superinfections have been well studied in the last few years, little is known about viral and fungal superinfections. Bacterial superinfections generally occur within the first seven days after influenza virus infection, and these lead to more fulminant disorders, combined with pulmonary inflammation and elevated mortality (24, 25). In some cases, however, bacterial superinfection occurs only when the viral infection appears to have been cured. There have been multiple descriptions to the effect that influenza virus infection paves the way for bacterial superinfection. As a result of an influenza virus infection, the cleaning function of the cilia is destroyed, the mucous membrane layer is dissolved and hence additional receptors for bacteria are exposed. Moreover, the immune response is dysregulated, which leads to reduced defense and increased inflammation processes (4, 26).

It has also been reported in the last few years that patients with severe influenza virus infection developed invasive pulmonary aspergillosis, which was caused by a superinfection by influenza viruses and fungi such as Aspergillus spp., especially Aspergillus fumigatus. This led to an even higher mortality rate compared to a simple infection with influenza viruses (27, 28).

Aspergillus spp. are filamentous, saprotrophic fungi that occur in the air and in the soil (11, 29). In healthy humans, the inhalative conidia are combated by mucociliary clearance and early immune defense mechanisms (11). In the case of patients with a weakened immune system or transplant patients, there is an elevated risk of complications and an increased mortality rate (5, 28). The likelihood of development of invasive pulmonary aspergillosis, which is characterized by the penetration of hyphae of Aspergillus fumigatus into the pulmonary tissue is further increased in patients with a weakened immune system and is associated with elevated mortality (10). A relatively new and unresearched clinical entity is post-influenza aspergillosis, which is difficult to diagnose. It recently became clear that influenza A and B virus infections are responsible for the evolution of superinfections with fungi in patients with and without weakened immune systems (6, 30, 31). It is assumed that, similarly to the case of bacterial superinfections, the loss of ciliary function of the mucous membrane means that the patients are predisposed to superinfection with fungi and development of an invasive fungal infection (31).

The most efficient route to date for protection from annual influenza virus epidemics is vaccination (1, 13). Even though new vaccines are produced every year, the vaccination rate is low, efficiency is variable, and adapted vaccines are not available in good time in the event of entirely new occurrence of influenza A virus subtypes. New vaccines have to be adapted and produced for each new virus variant, which takes at least six months. There is thus a period between the occurrence of a pathogen and the introduction of the new vaccine in which the population is unprotected. Accordingly, it is necessary to use antiviral alternatives for infection control.

For antiviral therapy, the European Medicines Agency (EMA) has approved three classes of compounds that target either the ion channel protein (M2), neuraminidase or CAP-dependent endonuclease. While neuraminidase inhibitors are effective against influenza A and B viruses, M2 inhibitors are ineffective against influenza B viruses (13). Regrettably, influenza viruses rapidly develop resistances against therapeutics. Several reports refer to resistant variants of influenza B viruses against oseltamivir, and influenza A viruses of the H5N1 type and the pandemic H1N1v types. The frequent occurrence of resistant variants in clinical isolates against adamantanes led to the recommendation not to use M2 inhibitors for treatment and prophylaxis until susceptibility to these medicaments has been reestablished in circulating influenza A viruses (16). A further weak point in the existing antiviral therapeutic approaches is that antiviral treatment has to be initiated as quickly as possible after the symptoms have commenced.

Novel antiviral strategies include (a) inhibition of virus-supporting cell functions, (b) promotion of antiviral defense or (c) alleviation of inflammation (32).

The most promising antiviral strategies for combating influenza infections are based on the fact that influenza viruses, as intracellular pathogens, are greatly dependent on cellular signal machinery. Influenza viruses are capable of manipulating cellular factors for their own purposes in order to ensure that they replicate and spread. Furthermore, influenza viruses can counteract the innate immune response of their hosts. In view of these dependences, cellular virus-supporting functions are targets for antiviral interventions (32). In the last few years, it has been possible to identify various cellular factors as suitable targets for antiviral intervention (33), including the Raf/MEK/ERK mitogen kinase cascade (32, 34-39), the IKK/NFκB module (40-42) and the PI3K signaling pathway (33, 43-48). Attacking most of these factors was found to be effective against influenza virus infection in vitro, but also in in vivo mouse models. The first clinical studies of LASAG suggest antiviral action thereof against influenza virus infection in hospitalized patients (61). Attacking cellular rather than viral factors reduces the likelihood of development of resistances since it is more difficult for the pathogen to compensate for the lack of cellular function.

Among the antimycotics, triazoles are the first choice against aspergillosis, but echinocandin and the polyene amphotericin B are also used (29). The most recent development of resistance in Aspergillus spp., especially in Aspergillus fumigatus variants, is concerning.

There has been a global increase in azole resistance and, consequently, new guidelines recommend a voriconazole-eninocandin combination or amphotericin B (5). In general, preventative treatment of aspergillosis is not initiated. Only when infection with fungi is obvious or has been detected is empirical antifungal therapy initiated. This is a combination of immunomodulatory treatment and antimycotic therapy (29).

There are only limited options for the treatment of viral/fungal superinfections. In fact, corticosteroid treatment, which is frequently used in ER, is contraindicated owing to prolonged virus excretion with elevated mortality and increased invasive pulmonary aspergillosis (5). Since more recent studies indicate significant interference by Aspergillus fumigatus and its host during invasive aspergillosis, specific fungi-assisting cellular factors and fungally induced harmful inflammation processes are alternative attack options (29, 49, 50).

Because of the phylogenetic relationship between fungi and humans, there are various factors that are very conserved (51). For instance, it would be possible for these homologs to be attacked simultaneously by a chemical inhibitor that subsequently acts on the host cell, Aspergillus spp., or pathogen-induced cell functions. Among these, the mitogen-activated protein kinase (MAPK) cascades have been identified (49, 52-54), which regulate fungal processes such as biofilm formation (55), stress tolerance (52, 53), virulence (49), and host cell functions such as inflammation processes (50). Interestingly, it has recently been shown that antimycotics such as itraconazole have an inhibiting effect on influenza virus replication (56, 57) and SARS-CoV-2 infection (58). Even though the effects are likely to be attributable to different mechanisms of action of the compound during the replication of the different viruses, in the case of influenza virus infection, there has been discussion of blockaded cholesterol export from the endolysosomal compartment and hence reduced replication (56).

Remarkably, MAPK have also been identified as potential targets for influenza virus intervention, which are involved in viral replication and virus-induced hyperinflammation (59, 60).

There is therefore a continuing need for novel therapeutics for treatment of influenza viruses, Aspergillus spp., and especially of superinfection by influenza viruses and Aspergillus Spp.

### SUMMARY OF THE INVENTION

The invention relates to an amine (AM) of the general formula (I)

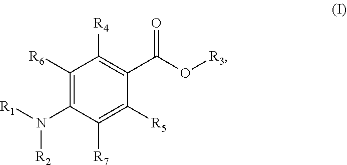

where, in formula (I),
R₁ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₂ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₃ is —(CH₂)ₙNR₃R₉;
n is 1 to 5, preferably 1 to 3, more preferably 1 to 2,
R₃ is (C₁₋₁₀)alkyl, preferably (C₁₋₂)alkyl, R₉ is (C₁₋₁₀)alkyl, preferably (C₁₋₂)alkyl;
R₄ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or halogen;
R₅ is H, halogen, (C₁₋₁₀)alkyl, (C₂₋₁₀)alkenyl, (C₅-C₁₄)aryl, (C₅-C₁₀)heteroaryl or —O—(C₁₋₁₀)alkyl, preferably H, halogen, (C₁₋₁₀)alkyl or —O(C₁₋₁₀)alkyl, more preferably H or halogen;
R₆ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
R₇ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O—(C₁₋₁₀)alkyl;
where the amine of formula (I) may optionally also be used in the form of a salt,
for use in treatment of infections with Aspergillus fumigatus and of superinfections with influenza viruses and Aspergillus spp.

In a further aspect, the invention relates to a carbonic acid adduct (KA) comprising at least one structural element of the general formula (II), (III) and/or (IV)

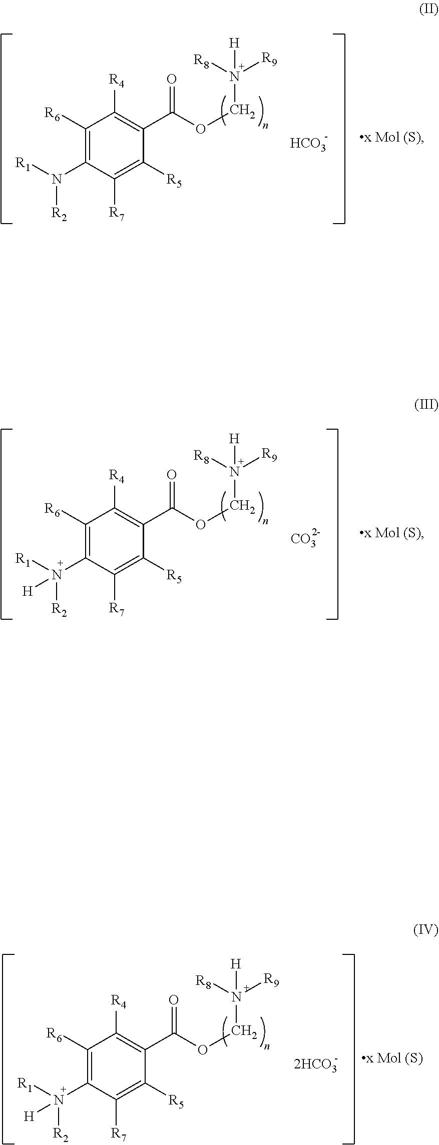

where, in formulae (II), (III) and (IV),
R₁ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₂ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₃ is —(CH₂)ₙNR₃R₉;
n is 1 to 5, preferably 1 to 3, more preferably 1 to 2,
R₃ is (C₁₋₁₀)alkyl, preferably (C₁₋₂)alkyl,
R₉ is (C₁₋₁₀)alkyl, preferably (C₁₋₂)alkyl;
R₄ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or halogen;
R₅ is H, halogen, (C₁₋₁₀)alkyl, (C₂₋₁₀)alkenyl, (C₅-C₁₄)aryl, (C₅-C₁₀)heteroaryl or —O—(C₁₋₁₀)alkyl, preferably H, halogen, (C₁₋₁₀)alkyl or —O(C₁₋₁₀)alkyl, more preferably H or halogen;
R₆ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
R₇ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
x is 0.5 to 30;
(S) is a salt;
for use in treatment of infections with Aspergillus fumigatus and of superinfections with influenza viruses and Aspergillus spp.

In a further aspect, the invention relates to a carbonic acid adduct (KA) comprising carbonic acid, at least one amine (AM) of the general formula (I) and at least one salt (S),

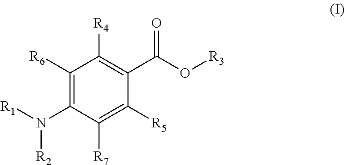

where, in formula (I),
R₁ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₂ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₃ is —(CH₂)ₙNR₈R₉;
n is 1 to 5, preferably 1 to 3, more preferably 1 to 2,
R₃ is (C₁₋₁₀)alkyl, preferably (C₁₋₂)alkyl,
R₉ is (C₁₋₁₀)alkyl, preferably (C₁₋₂)alkyl;
R₄ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or halogen;
R₅ is H, halogen, (C₁₋₁₀)alkyl, (C₂₋₁₀)alkenyl, (C₅-C₁₄)aryl, (C₅-C₁₀)heteroaryl or —O—(C₁₋₁₀)alkyl, preferably H, halogen, (C₁₋₁₀)alkyl or —O(C₁₋₁₀)alkyl, more preferably H or halogen;
R₆ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
R₇ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
where the at least one amine of formula (I) may optionally also be used in the form of a salt; preparable by a process comprising the steps of:
a) providing a solution (A) comprising at least one solvent, and CO₂ dissolved in the at least one solvent,
optionally b) dissolving a base (BA) that does not correspond to the amine (AM) in solution (A) to obtain solution (A1),
c) dissolving the at least one amine (AM) in solution (A) or (A1) to obtain solution (B),
d) freezing the solution obtained on conclusion of step c),
e) storing the solution frozen in step d) at −100 to 0° C. for no longer than 4 days; for use in treatment of infections with Aspergillus fumigatus and of superinfections with influenza viruses and Aspergillus spp.

The invention further relates to a pharmaceutical composition comprising the amine, or the carbonic acid adduct, for use in treatment of infections with Aspergillus fumigatus and of superinfections with influenza viruses and Aspergillus spp.

The substances of this underlying invention disclosure are capable of producing influenza virus infections and influenza virus-induced cytokine expression and of blocking hyphae formation by Aspergillus fumigatus. The present invention thus offers a new approach for antipathogenic therapy of both pathogens with a single active ingredient. Furthermore, potential influencing of pathogen-induced cellular factors enables regulation of excessive immune responses in superinfection scenarios.

### BRIEF DESCRIPTION OF THE FIGURES

FIG. 1: Procaine-containing active ingredients (e.g. procaine hydrochloride (HCl)) reduces hyphae formation by A. fumigatus.

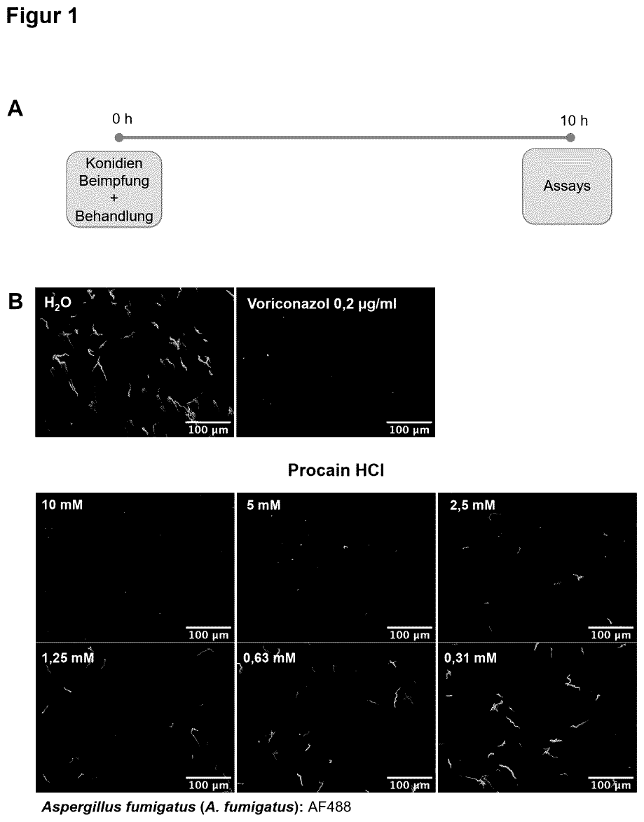

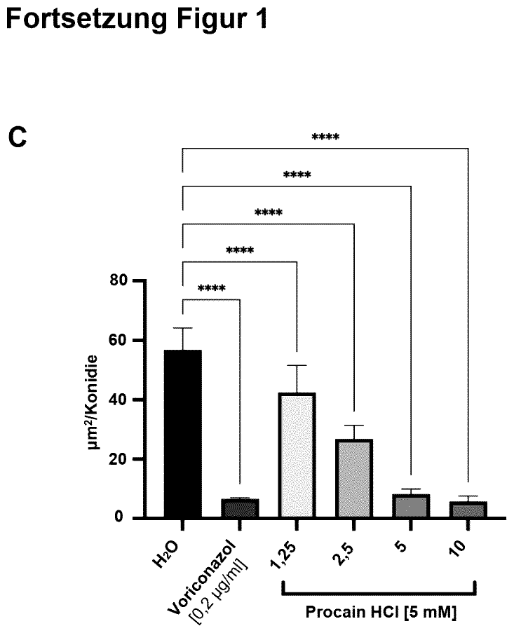

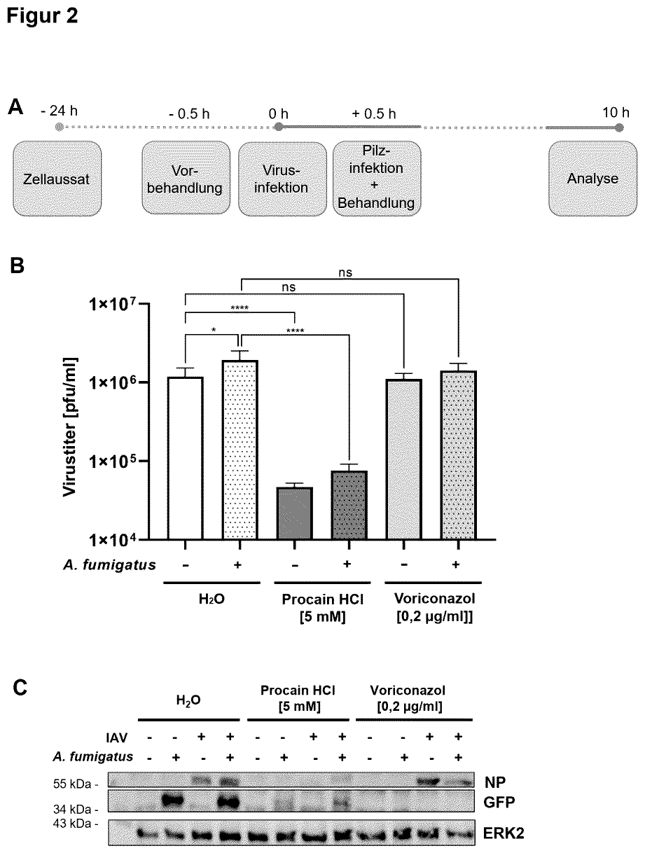

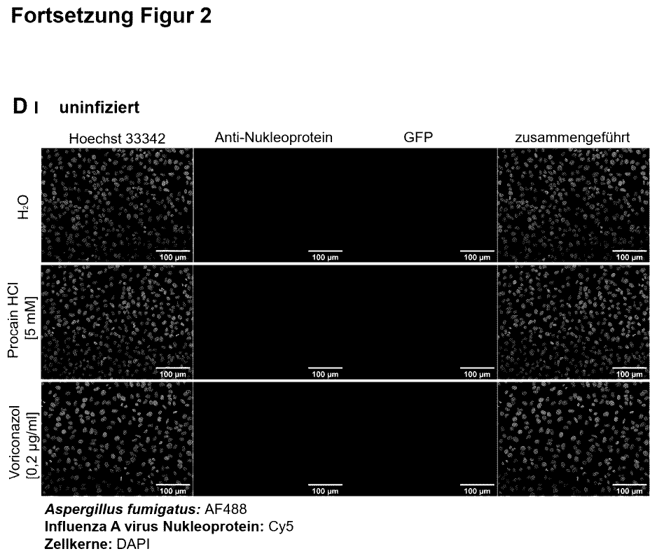

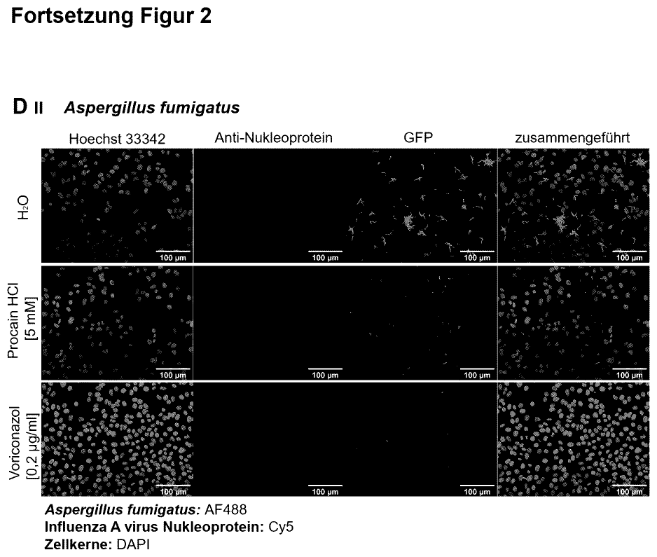

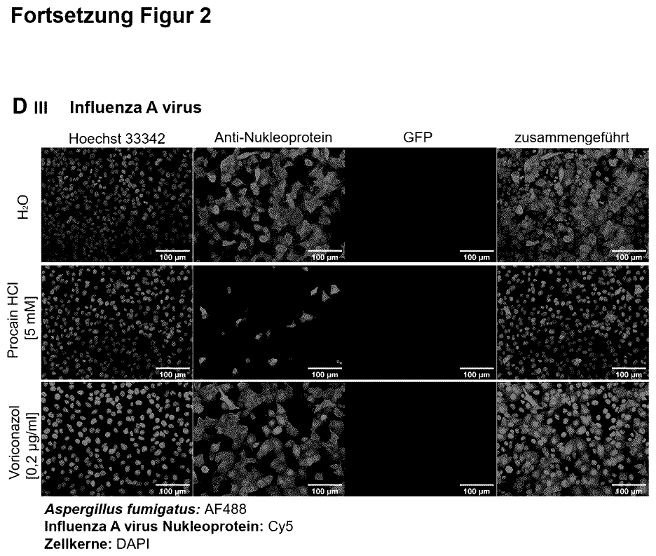

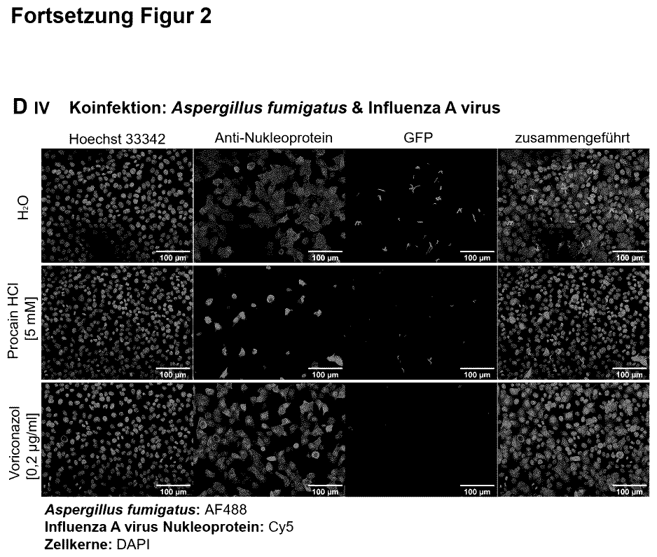

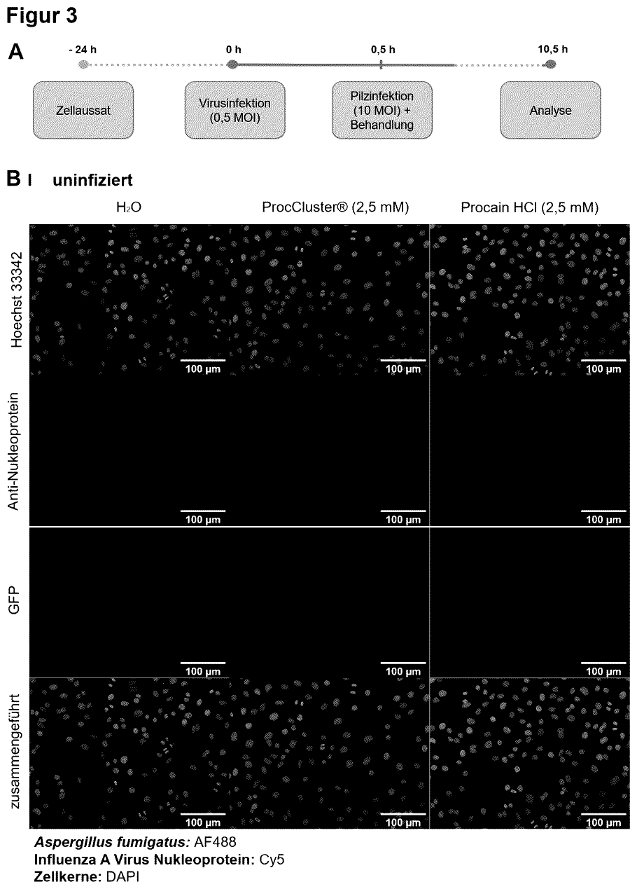

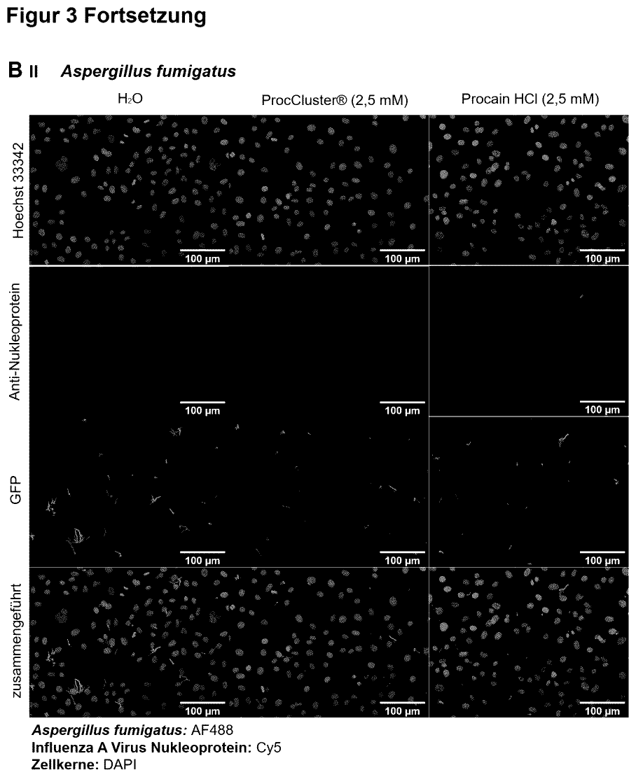

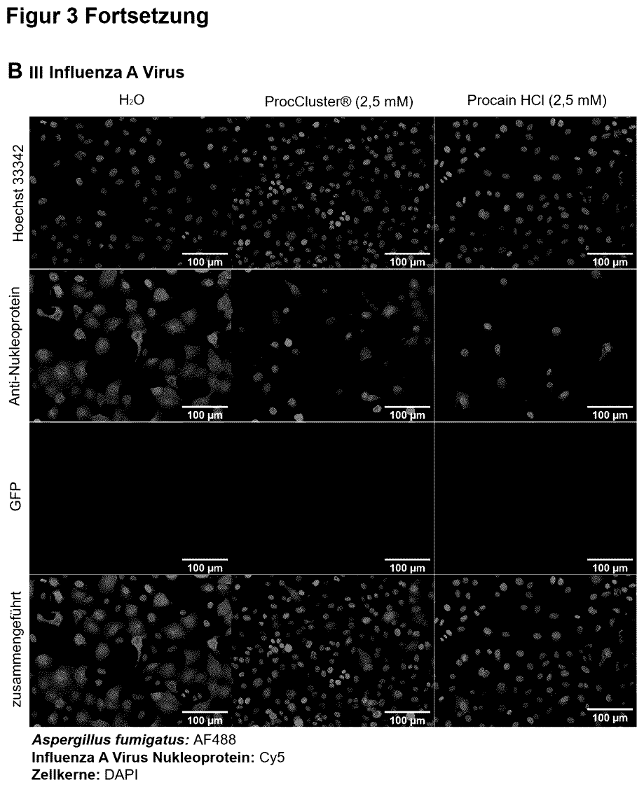

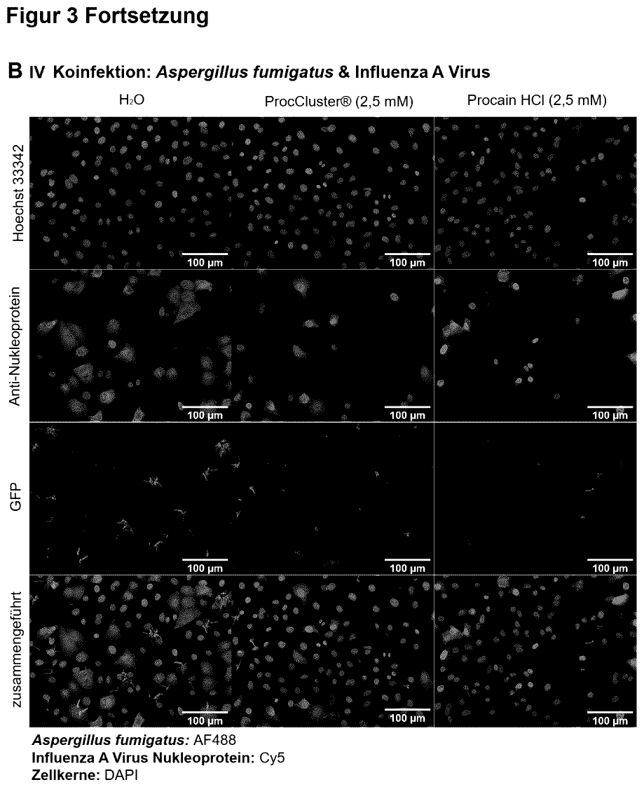

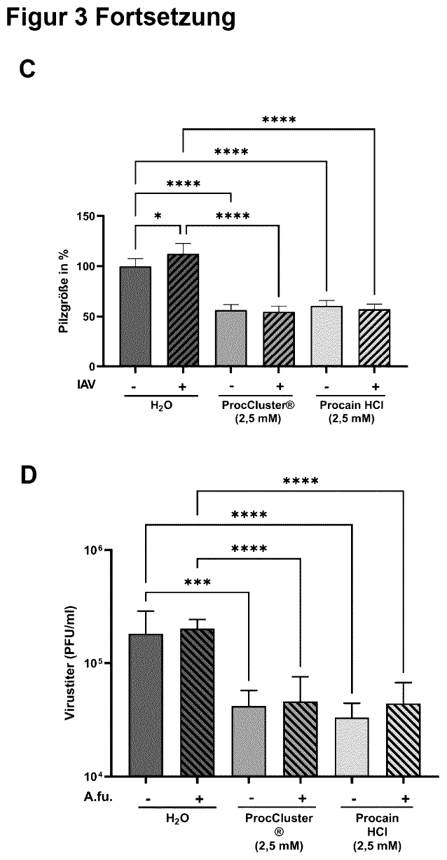

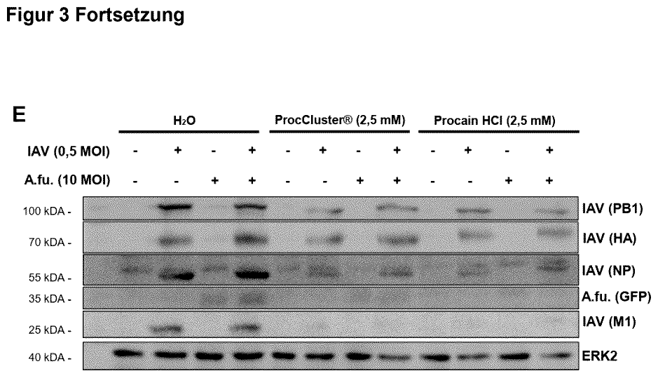

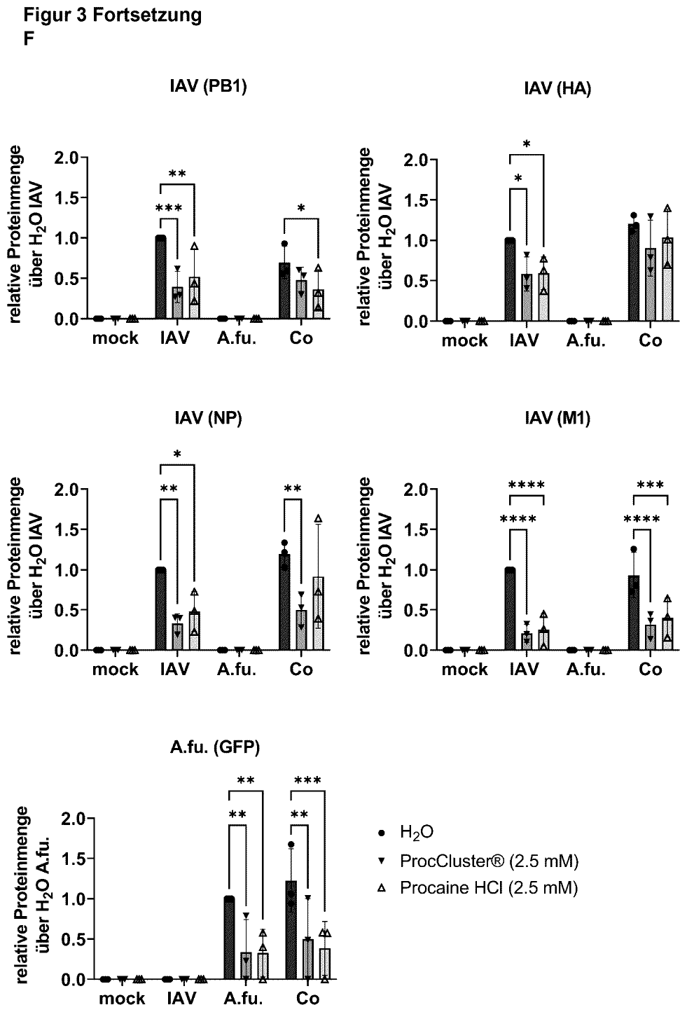

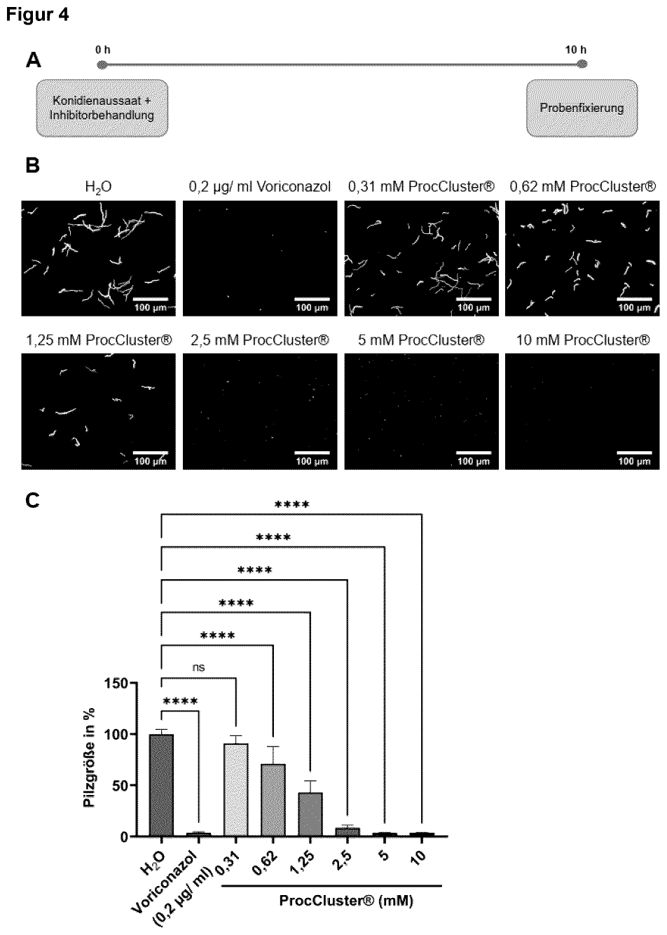

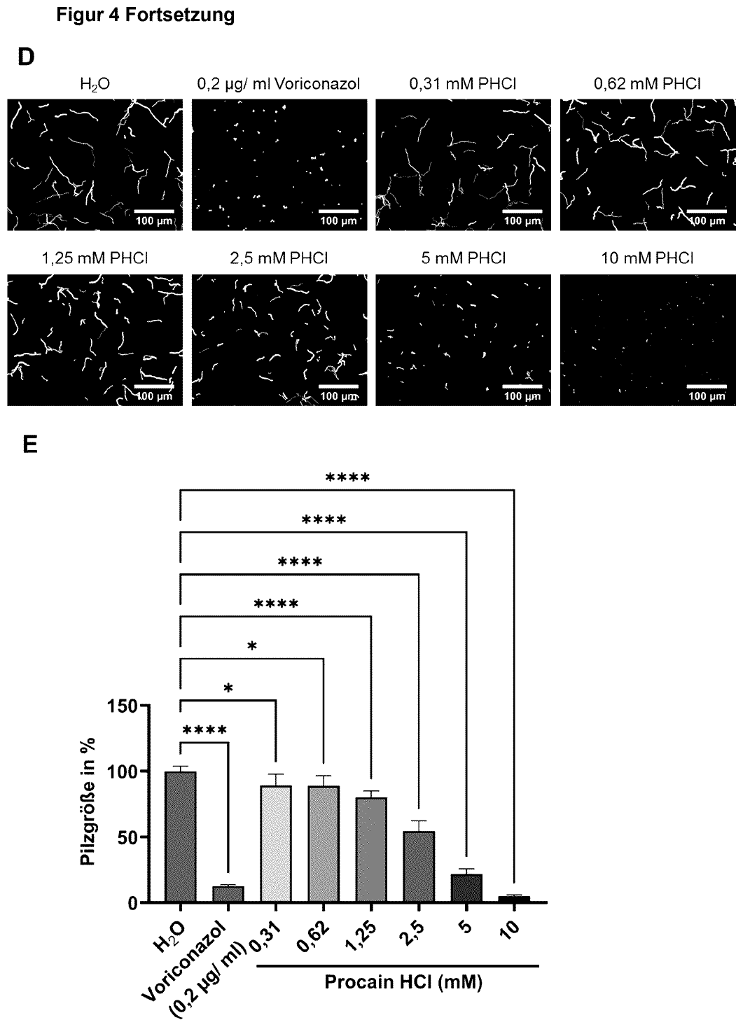

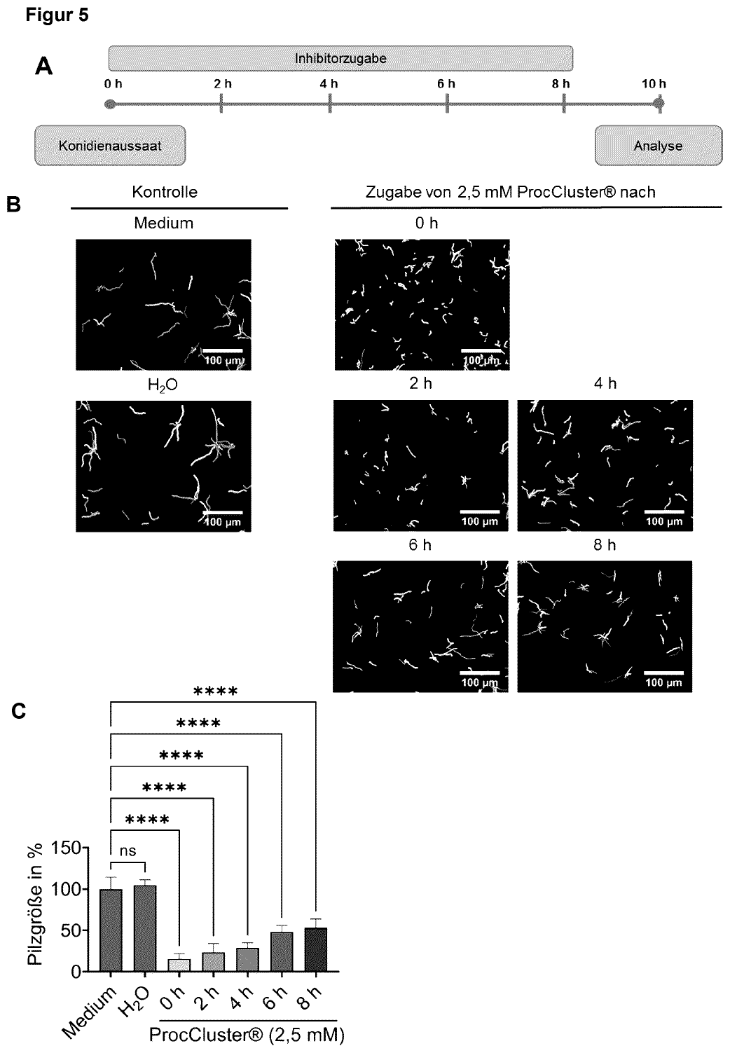

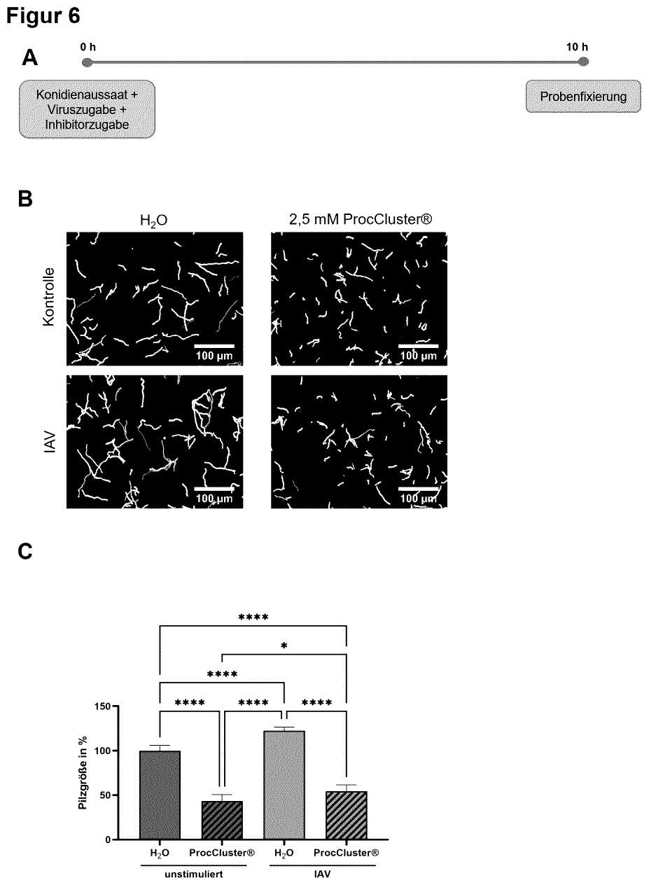

(A) Procedure for the treatment method: conidia of a green-fluorescence protein (GFP)-expressing A. fumigatus strain [5*10⁶ conidia/well] were sown on cover slips in 24-well plates in 300 μl of DMEM/BA medium (0.2% BSA, 1 mM MgCl₂ and 0.9 mM CaCl₂) and, in each case supplemented with solvent (H₂O), procaine HCl [0.31 (B) or 1.25 (C)—10 mM] or voriconazole [0.2 μg ml⁻¹], incubated at 37° C., 5% CO₂, for 10 h. Subsequently, the fungi were fixed (3.7% formaldehyde in PBS, 30 min, room temperature) and applied to microscope slides with the aid of fluorescent dye solution (Agilent, Santa Clara, CA, USA). The hyphae size was analyzed by (B) Axio Observer.Z1 microscope (Zeiss, Jena, Germany) and the Fiji V 1.52b software (github.com/fiji/fiji). (C) Fungus size was calculated with the particle counter software (github.com/chko90/particle counter). The data are a representation of four experiments (B) or the mean+SD of four experiments with two technical samples in each case (C). Statistical significance was calculated with GraphPad Prism 9.2 (Graphpad Software, Boston, USA) using the one-way ANOVA Dunnett multiple comparison test. P values are indicated by asterisks *p<0.05.

FIG. 2: The pathogenic load of influenza viruses and A. fumigatus is reduced in in vitro assays in the presence of procaine-containing active ingredients (e.g. procaine HCl). (A) Procedure for the treatment method: human lung epithelial cells (A549) [0.5*10⁵ cells/well] were sown in DMEM (10% FCS) on cover slips in 24-well plates and incubated at 37° C., 5% CO₂, for 23.5 hours. 30 min prior to infection, the cells were pretreated with the specified concentrations of procaine HCl or voriconazole in DMEM/BA (0.2% BSA, 1 mM MgCl₂ and 0.9 mM CaCl₂) then infected for 30 min with influenza virus A/Puerto Rico/8/34 [0.5 multiplicity of infection (MOI)] in PBS/BA (0.2% BSA, 1 mM MgCl₂ and 0.9 mM CaCl₂). Thereafter, the inoculum was removed, cells were washed with PBS, and conidia of A. fumigatus [10 MOI] were added in the presence of the specified substances in DMEM/BA/trypsin (0.2% BSA, 1 mM MgCl₂ and 0.9 mM CaCl₂, 167 ng ml⁻¹ trypsin-TPCK). Subsequently, the cells were incubated at 37° C., 5% CO₂, for 10 h. (B) Supernatants of infected cells were collected and viral titers (plaque-forming units (PFU)/ml) were examined by standard plaque titration. (C) Expression of viral nucleoprotein (NP), GFP of the A. fumigatus strain (GFP) and the cellular protein extracellular signal-regulated kinase 2 (ERK2) were visualized in Western Blot analyses (D_{I-IV}). The cells were fixed (3.7% formaldehyde in PBS, 30 min, room temperature) and prepared for immunofluorescence studies. The influenza virus nucleoprotein was visualized by means of an anti-influenza virus nucleoprotein antibody (BioRad) and AlexaFluor647 (Invitrogen). The cell nuclei were stained with bisbenzimide H-33342 trihydrochloride (Hoechst-33342; Merck), diluted 1:1000 in PBS (3% normal goat serum). The presence of A. fumigatus was visualized via internal GFP expression (GFP). The samples were applied to microscope slides with fluorescent dye solution (Agilent, Santa Clara, CA, USA). Imaging and image processing were effected with an Axio Observer.Z1 microscope (Zeiss, Jena, Germany) and the Fiji V 1.52b software (github.com/fiji/fiji). What are shown are the images of the individual channels and the superimposition thereof (combined). The data are the mean+SD of four independent experiments, including two technical samples (B), and an illustrative diagram from four experiments (C, D_{I-IV}). Statistical significance was verified with GraphPad Prism 9.2 (Graphpad Software, Boston, USA) using the multiple comparison test of one-way ANOVA with a Tukey analysis (p<0.05 was considered to be the level of statistical significance).

FIG. 3: The treatment of influenza A virus-infected and/or A. fumigatus-infected A549 cells with ProcCluster® or procaine HCl leads to a reduced virus load and hyphae formation 10 h after infection (p.i.). (A) Procedure for the treatment method: human lung epithelial cells (A549) [0.25*10⁵ cells/24 wells (B_{I-IV}-C) on cover slips or 0.5*10⁵ cells/12 wells (D-F)] were sown in DMEM (10% FCS) and incubated at 37° C., 5% CO₂, for 24 h. The cells were infected with influenza virus A/Puerto Rico/8/34 (IAV) [0.5 MOI] in PBS/BA (0.2% BSA, 1 mM MgCl₂ and 0.9 mM CaCl₂) for 30 min. Thereafter, the inoculum was removed, and conidia of A. fumigatus (A.fu.) [10 MOI] in the presence of ProcCluster®, procaine HCl or solvent control (H₂O) in DMEM/BA/trypsin (0.2% BSA, 1 mM MgCl₂ and 0.9 mM CaCl₂, 167 ng ml⁻¹ trypsin-TPCK) were added. Subsequently, the cells were incubated at 37° C., 5% CO₂, for 10 h (B_{I-IV}). The cells were fixed (3.7% formaldehyde in PBS, 1 h, room temperature) and prepared for immunofluorescence studies. The influenza virus nucleoprotein was visualized by means of an anti-influenza virus nucleoprotein antibody (BioRad) and AlexaFluor647 (Invitrogen). The cell nuclei were stained with bisbenzimide H-33342 trihydrochloride (Hoechst-33342, Merck). The presence of A. fumigatus was visualized via internal GFP expression. The samples were applied to microscope slides with fluorescent dye solution (Agilent, Santa Clara, CA, USA). The scale of the immunofluorescence images corresponds to 100 μm. Immunofluorescence detection and imaging and image processing were effected with an Axio Observer.Z1 microscope (Zeiss, Jena, Germany) and the Fiji V 1.52b software (github.com/fiji/fiji) (C). Fungus size was calculated with the particle counter software (github.com/chko90/particle counter). Five immunofluorescence images were recorded for each sample. The fungus sizes ascertained were normalized and the virus-uninfected, untreated, A. fumigatus-infected control was set to 100%. (D) Supernatants of IAV- or co-infected A549 cells were collected 10 h after infection. Virus titers were examined by standard plaque titration and are shown as PFU/ml. (E) Expression of viral polymerase basic protein 1 (PB1), hemagglutinin (HA), nucleoprotein (NP) and matrix protein 1 (M1) and GFP of A. fumigatus (GFP) and the housekeeping protein ERK2 were visualized by Western Blot analysis. (F) The individual signals from the proteins were quantified in relation to ERK2 using the Fiji V1.52b software (github.com/fiji/fiji) and depicted as the relative amount of protein based on H₂O IAV or H₂O A.fu. The data are the mean+SD of three (C-E, F) independent experiments with technical duplicates. The immunofluorescence images (B) and the Western Blot images (E) are an experiment representative of three experiments (B, E) with technical duplicates (B). Statistical significance was ascertained by a one-way (C, D) or two-way (F) ANOVA multiple comparison test with a Tukey analysis using GraphPad Prism 9.2 (Graphpad Software, Boston, USA) (p<0.05 was considered to be the level of statistical significance).

FIG. 4: Treatment of A. fumigatus with ProcCluster® or procaine HCl in a human cell-free system leads to reduced hyphae formation. (A) Procedure for the treatment method: conidia of a GFP-expressing A. fumigatus strain [5*10⁶ conidia/well] was sown on cover slips in 24-well plates and incubated with solvent (H₂O) or with the specified concentrations of voriconazole, ProcCluster® (B-C) or procaine HCl (D-E) in DMEM with 10% FCS at 37° C., 5% CO₂, for 10 h. Subsequently, the fungi were fixed (3.7% formaldehyde in PBS, 1 h, room temperature) and applied to microscope slides with fluorescent dye solution (Agilent, Santa Clara, CA, USA). (B, D) The growth of A. fumigatus was visualized via internal GFP expression. The scale of the immunofluorescence images corresponds to 100 μm. Detection of immunofluorescence and imaging and image processing were effected with an Axio Observer.Z1 microscope (Zeiss, Jena, Germany) and the Fiji V 1.52b software (github.com/fiji/fiji). (C, E) Fungus size was determined by means of the particle counter software (github.com/chko90/particle counter). Five immunofluorescence images were taken for each sample. The fungus sizes ascertained were normalized and the solvent control was set at 100%. Statistical significance was ascertained by a one-way ANOVA test with Dunnett multiple comparison test using GraphPad Prism 9.2 (Graphpad Software, Boston, USA) (p<0.05 was considered to be the level of statistical significance).

FIG. 5: Treatment of A. fumigatus with ProcCluster® in a human cell-free system, during germination, leads to a reduction in further hyphae growth. (A) Procedure for the treatment method: conidia of a GFP-expressing A. fumigatus strain [5*10⁶ conidia/well] were sown on cover slips in 24-well plates and, at the times specified, incubated with 2.5 mM ProcCluster® or with solvent (H₂O) in DMEM with 10% FCS (added at time 0) at 37° C., 5% CO₂, for 10 h. Subsequently, the fungi were fixed (3.7% formaldehyde in PBS, 1 h, room temperature) and applied to microscope slides with fluorescent dye solution (Agilent, Santa Clara, CA, USA). (B) The growth of A. fumigatus was visualized via internal GFP expression. The scale of the immunofluorescence images corresponds to 100 μm. Immunofluorescence detection and imaging and image processing were effected with an Axio Observer.Z1 microscope (Zeiss, Jena, Germany) and the Fiji V 1.52b software (github.com/fiji/fiji). (C) Fungus size was determined with the particle counter software (github.com/chko90/particle counter). Five immunofluorescence images were taken for each sample. The fungus sizes ascertained were normalized and the untreated control (medium) was set to 100%. Statistical significance was ascertained by a one-way ANOVA test with Dunnett multiple comparison test using GraphPad Prism 9.2 (Graphpad Software, Boston, USA) (p<0.05 was considered to be the level of statistical significance).

FIG. 6: The treatment of Aspergillus fumigatus with ProcCluster® in a human cell-free system leads to inhibition of IAV-induced hyphae formation. (A) Procedure for the treatment method: conidia of a GFP-expressing A. fumigatus strain [5*10⁶ conidia/well] were sown on cover slips in 24-well plates and incubated with purified virus particles of influenza virus A/Puerto Rico/8/34 [10 IAV particles/fungus] and with solvent (H₂O) or 2.5 mM ProcCluster® in DMEM with 10% FCS at 37° C., 5% CO₂, for 10 h. Subsequently, the fungi were fixed (3.7% formaldehyde in PBS, 1 h, room temperature) and applied to microscope slides with fluorescent dye solution (Agilent, Santa Clara, CA, USA). (B) The growth of A. fumigatus was visualized via internal GFP expression. The scale of the immunofluorescence images corresponds to 100 μm. Immunofluorescence detection and imaging and image processing were effected with an Axio Observer.Z1 microscope (Zeiss, Jena, Germany) and the Fiji V 1.52b software (github.com/fiji/fiji). (C) Fungus size was determined with the particle counter software (github.com/chko90/particle counter). Five immunofluorescence images were taken for each sample. The fungus sizes ascertained were normalized and the unstimulated solvent control was set to 100%. Statistical significance was ascertained by a one-way ANOVA test with Dunnett multiple comparison test using GraphPad Prism 9.2 (Graphpad Software, Boston, USA) (p<0.05 was considered to be the level of statistical significance).

### DETAILED DESCRIPTION OF THE INVENTION

The invention is directed to an amine (AM) of the general formula (I)

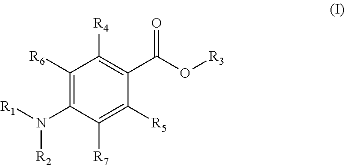

where, in formula (I),
R₁ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₂ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₃ is —(CH₂)ₙNR₈R₉;
n is 1 to 5, preferably 1 to 3, more preferably 1 to 2,
R₃ is (C₁₋₁₀)alkyl, preferably (C₁₋₂)alkyl,
R₉ is (C₁₋₁₀)alkyl, preferably (C₁₋₂)alkyl;
R₄ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or halogen;
R₅ is H, halogen, (C₁₋₁₀)alkyl, (C₂₋₁₀)alkenyl, (C₅-C₁₄)aryl, (C₅-C₁₀)heteroaryl or —O—(C₁₋₁₀)alkyl, preferably H, halogen, (C₁₋₁₀)alkyl or —O(C₁₋₁₀)alkyl, more preferably H or halogen;
R₆ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
R₇ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O—(C₁₋₁₀)alkyl;
where the amine of formula (I) may optionally also be used in the form of a salt,
for use in treatment of infections with Aspergillus fumigatus and of superinfections with influenza viruses and Aspergillus spp., preferably of superinfections with influenza viruses and Aspergillus spp.

The influenza viruses are preferably selected from the group consisting of influenza A viruses, influenza B viruses, and influenza C viruses, preferably influenza A viruses.

Aspergillus spp. is preferably Aspergillus fumigatus.

Treatment of infections with Aspergillus fumigatus in the context of the invention preferably means treatment of aspergillosis which is caused by infection with Aspergillus fumigatus.

A superinfection in the context of this invention means an infection of a subject (e.g. patient) first with one pathogen, followed with a time delay by infection with at least one second pathogen before the infection with the first pathogen has been overcome, such that the result is at least temporarily simultaneous infection with the first and with the second pathogen. Thus, in this context, infection first with influenza viruses, preferably influenza A viruses, may be followed by an additional infection with Aspergillus spp., preferably Aspergillus fumigatus or first with Aspergillus spp. followed by an additional infection with influenza viruses, preferably influenza A viruses, preferably first infection with influenza viruses, preferably influenza A viruses, followed by an additional infection with Aspergillus spp. Preferably, in the context of the superinfection, the infection with Aspergillus spp., preferably Aspergillus fumugatus, triggers aspergillosis in the infected subject. Preferably, in the course of the superinfection, the infection with influenza viruses triggers influenza in the infected subject.

Use in treatment of viral disorders, especially of influenza, comprises exploitation of an antiviral effect. Typically, active antiviral ingredients inhibit the development and propagation cycle rather than directly attacking the viruses.

Without being tied to any particular theoretical explanation, it is currently assumed that the observed antiviral effect of the compounds of the invention is likely to be mediated preferably indirectly by interaction with metabolic pathways of the infected organism that are responsible for propagation of the virus.

Use in treatment of fungal infections comprises exploitation of an antimycotic effect. Typically, antimycotic substances have a destructive effect on the cell wall of the fungi.

Without being tied to any particular theoretical explanation, it is currently assumed that the observed antimycotic effect of the compounds of the invention is likely to be mediated preferably indirectly by interaction with metabolic pathways of the fungus that are responsible for vitality.

In one embodiment, in formula (I), R₁, R₂, R₃, R₄, R₅, R₆ is H;
R₃ is —(CH₂)ₙNR₈R₉;
n is 1 to 5, preferably 1 to 3, more preferably 1 to 2,
R₃ is (C₁₋₁₀)alkyl, preferably (C₁₋₂)alkyl,
R₉ is (C₁₋₁₀)alkyl, preferably (C₁₋₂)alkyl.

In the present invention, definitions such as (C₁₋₁₀)alkyl, as defined, for example, for the R₁ radical of formula (I), mean that this substituent (radical) is a saturated alkyl radical having a carbon number from 1 to 10. The alkyl radical may be either linear or branched, and optionally cyclic. Alkyl radicals having both a cyclic and a linear component are likewise covered by this definition. The same applies to other alkyl radicals, for example a C₁-2 alkyl radical. Alkyl radicals may optionally also be mono- or polysubstituted by functional groups such as amino, hydroxyl, halogen, aryl or heteroaryl. Unless stated otherwise, alkyl radicals preferably have no functional groups as substituents. Examples of alkyl radicals are methyl, ethyl, n-propyl, n-butyl, n-pentyl, n-hexyl, n-heptyl, n-octyl, n-nonyl, isopropyl (also called 2-propyl or 1-methylethyl), isobutyl, tert-butyl, sec-pentyl, neopentyl, 1,2-dimethylpropyl, isoamyl, isohexyl, isoheptyl.

In the present invention, definitions such as C₂₋₁₀ alkenyl, as defined, for example, for the R₁ radical of formula (I), mean that this substituent (radical) is an alkenyl radical having a carbon number from 2 to 10 and having at least one unsaturated carbon-carbon bond. The alkenyl radical may be either linear or branched and optionally cyclic. Alkenyl radicals having both a cyclic and a linear component are likewise covered by this definition. The same applies to other alkenyl radicals, for example a C₂₄ alkenyl radical. Alkenyl radicals may optionally also be mono- or polysubstituted by functional groups such as amino, hydroxyl, halogen, aryl or heteroaryl. Alkenyl radicals preferably do not have any further functional groups as substituents. Examples of alkenyl radicals are vinyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 3-heptenyl, 4-heptenyl, 1-octenyl, 3-octenyl, 5-octenyl, 1-nonenyl, 2-nonenyl.

In the present invention, the definition aryl means an aromatic or heteroaromatic radical. An aromatic radical is an aromatic cyclic hydrocarbon that may consist of a ring or a ring system composed of two or more fused rings. The aromatic radical may, for example, be monocyclic, bicyclic or tricyclic. The monocyclic aromatic radical preferably forms a 5- or 6-membered ring. The bicyclic aromatic ring preferably forms a 9- or 10-membered ring. The tricyclic aromatic ring preferably forms a 13- or 14-membered ring. A definition such as (C₅-C₁₄)aryl means that the aryl group comprises 5 to 14 carbon atoms. The aryl group preferably contains 3 to 14, more preferably 4 to 6, carbon atoms. Aryl radicals may optionally also be mono- or polysubstituted by functional groups such as alkyl, alkenyl, amino, cyano, —CF₃, hydroxyl, halogen, aryl or heteroaryl; the aryl radicals preferably do not have any further substituents. Examples of aromatic radicals are phenyl and naphthyl.

In the present invention, the definition heteroaryl means a heteroaromatic radical. What is meant by “heteroaromatic ring” is that, in an aromatic radical as defined above, the ring system of which is formed by carbon atoms, one or more of these carbon atoms are replaced by heteroatoms such as O, N or S. A definition such as (C₅-C₁₀)heteroaryl is based on the corresponding definition for the aryl group, and means that the heteroaryl group has 5 to 10 atoms in the ring. However, as defined above, one or more carbon atoms are replaced by heteroatoms. This means that the (C₅-C₁₀)heteroaryl group has 5 to 10 atoms in the ring, but not all of these are carbon atoms. Therefore, for example, furanyl would be a C₅-heteroaryl group. Heteroaryl radicals may optionally also be mono- or polysubstituted by functional groups such as alkyl, alkenyl, amino, cyano, —CF₃, hydroxyl, halogen, aryl or heteroaryl; preferably, the heteroaryl radicals do not have any further substituents. Examples of heteroaromatic radicals that are covered by the definition of aryl in the present invention are furanyl, thienyl, oxazolyl, pyrazolyl, pyridyl and indolyl.

In the present invention, the definition halogen, as defined above, for example, for the R₄ radical for formula (I), means a chlorine, bromine, iodine or fluorine substituent. It is preferably a chlorine or fluorine substituent.

The amine (AM) of the general formula (I) is preferably selected from the group consisting of 2-(N,N-diethylamino)ethyl 4-aminobenzoate (procaine), ethyl 4-aminobenzoate (benzocaine), 2-(diethylamino)ethyl 4-amino-2-chlorobenzoate (chloroprocaine), 2-diethylaminoethyl 4-amino-3-butoxybenzoate (oxybuprocaine), 2-(dimethylamino)ethyl 4-(butylamino)benzoate (tetracaine), more preferably 2-(N,N-diethylamino)ethyl 4-aminobenzoate (procaine).

The amines (AM) of the general formula (I) and the amines (AM) specifically referred to above are in medical use as local anesthetics and are correspondingly commercially available.

The invention further relates to a carbonic acid adduct (KA) comprising at least one structural element of the general formula (II), (III) and/or (IV)

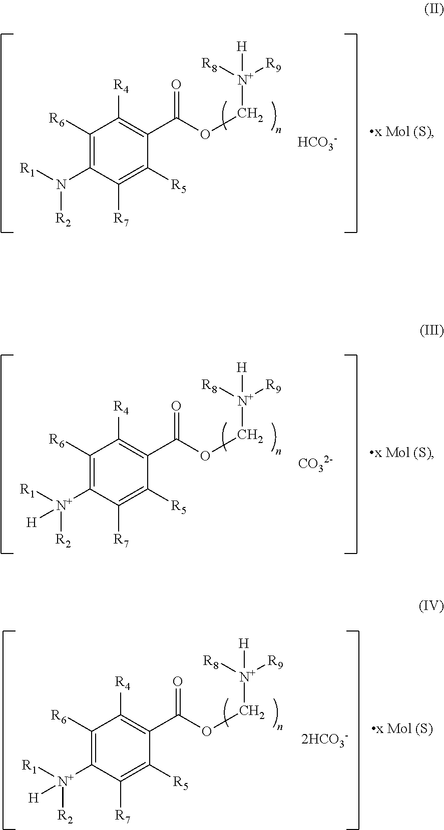

where, in formulae (II), (III), and (IV),
R₁ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₂ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₃ is —(CH₂)ₙNR₈R₉;
n is 1 to 5, preferably 1 to 3, more preferably 1 to 2,
R₃ is (C₁₋₁₀)alkyl, preferably (C₁₋₂)alkyl,
R₉ is (C₁₋₁₀)alkyl, preferably (C₁₋₂)alkyl;
R₄ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or halogen;
R₅ is H, halogen, (C₁₋₁₀)alkyl, (C₂₋₁₀)alkenyl, (C₅-C₁₄)aryl, (C₅-C₁₀)heteroaryl or —O—(C₁₋₁₀)alkyl, preferably H, halogen, (C₁₋₁₀)alkyl or —O(C₁₋₁₀)alkyl, more preferably H or halogen;
R₆ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
R₇ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
x is 0.5 to 30;
(S) is a salt;
for use in treatment of infections with Aspergillus fumigatus and of superinfections with influenza viruses and Aspergillus spp., preferably of superinfections with influenza viruses and Aspergillus spp.

In one embodiment, in formula (I), R₁, R₂, R₃, R₄, R₅, R₆ is H;
R₃ is —(CH₂)ₙNR₈R₉;
n is 1 to 5, preferably 1 to 3, more preferably 1 to 2,
R₃ is (C₁₋₁₀)alkyl, preferably (C₁₋₂)alkyl,
R₉ is (C₁₋₁₀)alkyl, preferably (C₁₋₂)alkyl.

The salt (S) comprises at least one cation selected from Na⁺, K⁺, Li⁺, Mg²⁺, Zn²⁺, Fe²⁺, Fe³⁺ and Mn²⁺, preferably Na⁺. The salt (S) further comprises at least one anion selected from Cl⁻, Br⁻, I⁻, F⁻, SO₄²⁻, SO₃²⁻, HSO₄⁻, HSO₃⁻, —HCO₃⁻, CO₃²⁻, PO₄³⁻, HPO₄²⁻, H₂PO₄⁻, SiO₄⁴⁻, AlO₂⁻, SiO₃⁻ and/or [AlO₂)₁₂(SiO₂)₂]²⁻, preferably Cl⁻ and Br⁻, more preferably Cl⁻.

The preparation of the carbonic acid adduct (KA) is also disclosed in WO2006/007835, DE 10 2013 015 035 A1 and WO2019/048590, to which reference is made here.

The invention further relates to a carbonic acid adduct (KA) comprising carbonic acid, at least one amine (AM) of the general formula (I) and at least one salt (S),

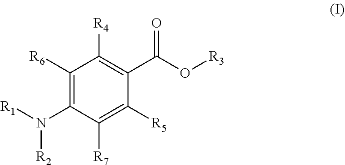

where, in formula (I),
R₁ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₂ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₃ is —(CH₂)ₙNR₈R₉;
n is 1 to 5, preferably 1 to 3, more preferably 1 to 2,
R₃ is (C₁₋₁₀)alkyl, preferably (C₁₋₂)alkyl,
R₉ is (C₁₋₁₀)alkyl, preferably (C₁₋₂)alkyl;
R₄ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or halogen;
R₅ is H, halogen, (C₁₋₁₀)alkyl, (C₂₋₁₀)alkenyl, (C₅-C₁₄)aryl, (C₅-C₁₀)heteroaryl or —O—(C₁₋₁₀)alkyl, preferably H, halogen, (C₁₋₁₀)alkyl or —O(C₁₋₁₀)alkyl, more preferably H or halogen;
R₆ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
R₇ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
where the at least one amine of formula (I) may optionally also be used in the form of a salt; for use in treatment of infections with Aspergillus fumigatus and of superinfections with influenza viruses and Aspergillus spp.

In one embodiment, in formula (I), R₁, R₂, R₄, R₅, R₆ is H;
R₃ is —(CH₂)ₙNR₈R₉;
n is 1 to 5, preferably 1 to 3, more preferably 1 to 2,
R₃ is (C₁₋₁₀)alkyl, preferably (C₁₋₂)alkyl,
R₉ is (C₁₋₁₀)alkyl, preferably (C₁₋₂)alkyl.

The salt (S) may be formed, for example, by an acid-base reaction of the base (BA), in the case of performance of step b) with the acid added onto the amine (AM), if an acid addition salt of the amine (AM) is used. The salt (S) can also be added directly in one of steps a), b) and/or c). Direct addition of the salt (S) is preferred if the amine (AM) is not used in salt form and/or step b) is not conducted.

The carbonic acid adduct (KA) preferably remains stable when stored at a temperature of 2 to 10° C. for at least 12 months, more preferably for at least 13 months, even more preferably for at least 20 months, especially preferably for at least 23 months, and most preferably for at least 27 months.

The carbonic acid adduct (KA) is no longer considered to be stable when the specific bands of the at least one amine (AM) can be detected by IR spectroscopy, especially in the solid-state carbonic acid adduct (KA). The specific IR bands of the amine are those bands that are also detected in IR spectroscopy analysis of the pure amine (AM). If the amine (AM) is bound in the stable carbonic acid adduct (KA), the specific IR bands of the amine (AM) are not detected.

Loss of stability in individual embodiments may also be associated with an increase in pH, or with measurement of two melting/decomposition ranges, i.e. one range corresponding to the amine (KA) and one range corresponding to the carbonic acid adduct (KA). In the carbonic acid adduct that has become unstable as a result of at least partial breakdown to the amine (AM) and CO₂ and/or water, there may also be a change in dissolution characteristics. The carbonic acid adduct (KA) that has become unstable may be found to be sparingly soluble or at least partly incompletely dissolved.

The carbonic acid adduct (KA) is preparable by a process comprising the steps of a), optionally b), c), d) and e).

In step a), a solution (A) comprising at least one solvent and CO₂ dissolved in the at least one solvent is provided.

Solution (A) comprises at least one solvent and CO₂ in dissolved form. CO₂ in the context of this invention means carbon dioxide. CO₂ in dissolved form in the context of this invention means all forms of CO₂ that it enters into on dissolution. For example, it is known in respect of aqueous solutions that the dissolved CO₂ in the solution may be present, inter alia, in equilibrium as CO₂, as carbonic acid, as singly or doubly deprotonated carbonic acid, i.e. as hydrogencarbonate or carbonate.

Solution (A) is obtained by introducing CO₂ into the at least one solvent. The CO₂ may be introduced into the solvent in all suitable forms that are known to the person skilled in the art. Preference is given to introducing gaseous or frozen CO₂ in the form of dry ice, more preferably gaseous CO₂, into the solvent. The gaseous CO₂ or frozen CO₂ is preferably at least of food quality, more preferably of a quality suitable for pharmaceutical purposes. The gaseous and/or frozen CO₂ used preferably has a purity of at least 99.5%, even more preferably 99.9%.

What is preferably meant by “food quality” in the context of this invention is that the relevant provisions in German and European food law are satisfied. These preferably include Regulation (EC) No 852/2004 and Regulation (EC) No 178/2002, and also Regulation (EC) No 1333/2008 and Regulation (EU) 231/2012.

What is preferably meant by “pharmaceutical quality” is that the relevant provisions of the European Pharmacopoeia (Ph. Eur.) are satisfied.

The CO₂ may also be introduced under pressure, especially when gaseous CO₂ is being introduced into the solution. What is meant by “introducing under pressure” in this connection is that a pressure greater than atmospheric pressure, preferably greater than 1.01325 bar, is used. For this purpose, the introduction of CO₂ into the solvent can be effected in a vessel that isolates the solvent from the environment such that a pressure can be generated in the vessel, especially via introduction of CO₂ above atmospheric pressure, preferably above 1.01325 bar. The introduction of the CO₂ into the solution, especially in gaseous form, can be effected in one step or at intervals.

The person skilled in the art is able to use any suitable solvent in step a). The solvent used is preferably a polar protic solvent; the solvent is more preferably water. Depending on the intended end use of the carbonic acid adduct (KA), the solvent may be used in various purities. For example, it is possible to use water of “Aqua ad iniectabilia” purity if the carbonic acid adduct (KA) is to be used for pharmaceutical and medical purposes.

Step a) may comprise component step a1), wherein the solvent, preferably prior to introduction of the CO₂, is cooled to 3 to 8° C., preferably to 5° C. The cooling can be effected by means of any methods known to the person skilled in the art that have been identified as being suitable. For example, the cooling can be effected by keeping the solvent in a refrigerator for a sufficiently long period of time for the solvent to have the target temperature. It is likewise possible, for example, to use external cooling.

Step a) may comprise component step a2), wherein the CO₂ is introduced into the solvent, preferably until attainment of a saturation concentration of 3 to 10 g/l, more preferably up to a saturation concentration of 4.5 to 7.5 g/l, based on the total volume of the solution. The pH of the solution after saturation with CO₂ is preferably ≤3.0 to ≤6.0, even more preferably ≤4.3 to ≤4.8. The CO₂ is preferably dissolved under pressure in component step a2), where the pressure is 1.5 to 10 bar, more preferably 1.9 to 7 bar, even more preferably 2 to 5 bar.

Step a) may comprise component step a3), wherein solution (A) which is preferably obtained in component step a2) is stored at 1 to 10° C. preferably for at least 30 min, more preferably for at least 50 min, even more preferably for at least 60 min; up to at most 5 days (120 h). Preferably, solution (A) which is preferably obtained in component step a2) is stored at 3 to 8° C. for at least 30 min, more preferably for at least 50 min, even more preferably for at least 60 min; up to at most 5 days (120 h).

Step a) preferably comprises all component steps a1), a2) and a3).

Preferably, component steps a1), a2) and a3) are performed in the sequence of a2) after a1), and a3) after a2).

It is optionally possible to conduct step b), wherein the base (BA) that does not correspond to the amine (AM) is dissolved in solution (A) to obtain solution (A1). The base (BA) is preferably a hydrogencarbonate or a carbonate, more preferably a hydrogencarbonate, even more preferably sodium hydrogencarbonate.

In step c) or c1), the at least one amine (AM) is dissolved in solution (A) or (A1) to obtain solution (B).

The at least one amine (AM), as defined above, may be used in step c) either in neutral form or in the form of a salt. Optionally, the at least one amine (AM) may also be used as a mixture of the neutral form of the amine (AM) with the salt form of the amine (AM). Therefore, the at least one amine (AM) may comprise the neutral amine (AM) and/or the salt form of the at least one amine (AM). The salt form of the at least one amine (AM) is preferably an acid addition salt; the acid addition salt is preferably a hydrochloride, hydrobromide, hydroiodide, hydrogensulfate, hydrogensulfite, hydrogenphosphate, hydromesylate, hydrotosylate, hydroacetate, hydroformate, hydropropanoate, hydromalonate, hydrosuccinate, hydrofumarate, hydroxalate, hydrotartrate, hydrocitrate, hydromaleate, more preferably a hydrochloride or hydrobromide, even more preferably a hydrochloride, of the at least one amine (AM).

Preferably, the concentration of the amine (AM) in solution (B) is 0.01 to 0.25 g/ml, preferably 0.03 to 0.20 g/ml, more preferably 0.08 to 0.15 g/ml.

Step c) may comprise component step c2), wherein the at least one amine (AM) is dissolved in solution (A), or, in the case of performance of step b), in solution (A1), to obtain solution (B).

In one possible embodiment, the ratio of the amine (AM) to the base (BA), in the case of performance of step b), in solution (B) is 2:1 to 5:1, more preferably 3:1 to 4:1, even more preferably 3.23:1 to 3.26:1 [g/g]].

In a further possible embodiment, the molar ratio of the amine (AM) to the base equivalents of the base (BA), in the case of performance of step b), in solution (B) is 0.8:1 to 1.5:1, preferably 1.2:1, more preferably 1:1. What is meant by base equivalents in this connection is that, when a monoacidic base is used, for example NaHCO₃, the molar ratio of the base (BA) to the amine (AM) conforms to the above-specified ratio. When a diacidic base (BA) is used, for example Na₂CO₃, based on the molar amount in moles of the base (BA) relative to the use of a monoacidic base, only half the amount of base is required to introduce the same amount of base equivalents. For example, in the case of a ratio of 1:1, when 10 mmol of amine (AM) is used, 10 mmol of NaHCO₃ is required, but only 5 mmol of Na₂CO₃.

In a further embodiment, step b) is performed and, in component step c1), the amine (AM) is added in the form of the acid addition salt, where the amine (AM) is added in such an amount with the acid bound thereto that the acid bound to the amine (AM) is capable of neutralizing the base (BA) to such an extent that solution (B) assumes a pH of 6 to 8.

Step c) may encompass component step c2), wherein solution (A) is added to solution (B) to obtain solution (B1).

Preferably, the concentration of the amine (AM) in solution (B1) is 0.01 to 0.25 g/ml, preferably 0.03 to 0.20 g/ml, more preferably 0.08 to 0.15 g/ml.

Step c) may comprise component step c3), wherein solution (B), or, in the case of performance of component step c2), solution (B1), is enriched with CO₂. Preferably, solution (B) is enriched with 2.5 g/I to 9 g/l, more preferably with 5 to 7.5 g/I CO₂.

Step c) may comprise component step c4), wherein solution (B), or, in the case of performance of component step c2), solution (B1), is stored at 1 to 10° C., preferably 3 to 8° C., for at least 1 h, preferably 24 h to 120 h, even more preferably 24 to 72 h.

Step c) may comprise component step c5), wherein solution (B), or, in the case of performance of component step b2), solution (B1), is enriched with CO₂, preferably to a total concentration of at least 6 g/l, more preferably at least 10 g/l, even more preferably at least 12 g/l, very especially preferably at least 14 g/I and most preferably at least 15 g/l. Preferably, in component step c5), a further 0.4 to 4.7 g/l, more preferably 1 to 3.5 g/l, of CO₂ is introduced or dissolved in solution (B) or (1) until the required total concentration is attained.

The expression “total concentration” relates here to the total concentration of dissolved CO₂ in solution (B) or (1), including the CO₂ bound in the carbonic acid adduct (KA). The total concentration is the result of adding up the increase in weight of the solution as a result of the CO₂ fed in in all the preceding enrichment steps a2) and/or c3), where they are performed, and c5), without taking account of CO₂ which is optionally added to the solution in the form of hydrogen carbonate or carbonate as base (BA).

The enriching of solution (B) or of solution (B1) in component step c5) with CO₂ to the required total concentration can be conducted at a pressure of 2.5 to 10 bar, preferably of 4 to 10 bar, more preferably of 5 to 10 bar, even more preferably at 6 to 10 bar, most preferably at 6.5 to 10 bar. Solution (B) or (B1), on enrichment with CO₂ in component step c5), is at a temperature of 3 to 8° C., more preferably of 5° C.

The enrichment of solution (B) or (B1) in component steps c3) and c5) can be effected in the same way as described for step a).

The pH of solution (B) or, in the case of performance of component step c2), of solution (B1), after performance of step c5) is s 7.0.

Step c) preferably comprises all component steps c1), c2), c3), c4) and c5).

Component steps c1), c2), c3), c4) and c5) are preferably performed in the sequence of c2) after c1), c3) after c2), c4) after c3), c5) after c4).

In step d), the solution obtained on conclusion of step c) is frozen. Preferably, in step d), solution (B), or, after performance of component step c2), solution (B1), is frozen.

The solution, preferably solution (B) or (B1), which is subjected to step d) has a CO₂ content of at least 6 g/l, preferably at least 10 g/l, more preferably at least 12 g/l, even more preferably at least 14 g/I and most preferably at least 15 g/l.

Preferably, the solution obtained on conclusion of step c), preferably solution (B) or (B1), is frozen at −100° C. to −20° C., more preferably at −90° C. to −30° C., even more preferably at −80 to −40° C. and most preferably at −70 to −50° C.

The freezing of the solution obtained on conclusion of step c), preferably solution (B) or (B1), can in principle be effected by any of the methods known to the person skilled in the art that have been identified as suitable. For example, the freezing can be effected by transferring the solution obtained in step c) to a suitable vessel immersed into a cooling medium. The vessel is preferably in bulb form. The vessel containing the solution obtained in step c) is preferably immersed into the cooling medium at an angle of 40°. The cooling medium may consist, for example, of a solvent such as methanol, ethanol or acetone, which is brought to the desired temperature by addition of dry ice, or suitable cooling apparatuses such as cryostats.

The freezing is preferably effected at atmospheric pressure, more preferably at 1.01325 bar.

Preferably, the solution obtained on conclusion of step c), preferably solution (B) or (1), is frozen within 0.3 to 60 minutes, more preferably within 1 to 30 minutes, even more preferably within 1.1 to 10 minutes, especially preferably within 1.5 to 5 minutes.

The solution obtained on conclusion of step c), preferably solution (B) or (1), is preferably frozen at a cooling rate of 10 to 100 K/min, more preferably at 20 to 80 K/min, even more preferably at 30 to 70 K/min and especially preferably at 40 to 60 K/min.

The vessel in which the solution obtained on conclusion of step c), preferably solution (B) or (1), is present during the freezing operation is preferably rotated in the cooling medium at 10 to 1000 rpm, more preferably at 50 to 600 rpm, even more preferably at 100 to 400 rpm and especially preferably at 200 to 300 rpm.

The freezing can be effected by the shell freezing method.

In step e), the solution frozen in step d), preferably solution (B) or (1), is stored at −100 to 0° C. for not longer than 4 days.

The solution frozen in step d), preferably solution (B) or (1), is preferably stored in step e) for 1.5 to 4 days, more preferably for 2.5 to 4 days.

The solution frozen in step d), preferably solution (B) or (1), is stored in step e) at −50 to 0° C., more preferably at −30 to −5° C., even more preferably at −25 to −10° C., especially preferably at −20 to −15° C.

The storage can in principle be effected at the defined temperature in any cooling device known to the person skilled in the art. For example, the storage can be conducted in an upright freezer or a freezer room.

The process by which the carbonic acid adduct (KA) is preparable may comprise a further step f) which is conducted after step e). In step f), the solution stored in step e), preferably solution (B) or (1), is dried to obtain dried carbonic acid adduct (KA).

Preferably, in step f), the water is removed from the solution stored in step e), preferably solution (B) or (1), down to a residual content of <0.8% by weight, more preferably down to a residual content of <0.1% by weight, based on the total weight of the dried carbonic acid adduct (KA).

Preferably, in step f), CO₂ not bound in the carbonic acid adduct (KA) is removed from the solution stored in step e), preferably (B) or (1), down to a residual content of <0.8% by weight, more preferably down to a residual content of <0.1% by weight, based on the total weight of the dried carbonic acid adduct (KA).

The drying can be effected by any of the methods known to the person skilled in the art that have been identified as suitable. The drying is preferably effected by freeze-drying, also called lyophilization. In the case of use of the freeze-drying method, step d) constitutes the freezing step, and step e) the maturing step.

Preferably, the pressure during the drying is 0.01 to 30 mbar, preferably 0.02 to 20 mbar, more preferably 0.03 to 10 mbar, even more preferably 0.03 to 0.5 mbar and most preferably 0.05 to 0.1 mbar. The pressure is preferably maintained throughout the drying operation. During the drying, the above-defined pressure is preferably attained within 7 h, more preferably within 5 h and especially preferably within 4 h from commencement of evacuation.

The endpoint of the drying can be ascertained by the person skilled in the art from the temperature progression recordings. The total drying time in step f) is preferably 10 to 60 h, more preferably 30 to 55 h, especially preferably 41 to 52 h. The total drying time is defined as the time span between the conclusion of the storage in step e) and the end of the drying in step D.

Preferably, the temperature throughout the drying in step f) is 0 to 20° C., preferably 4 to 18° C., more preferably 8 to 16° C.

The invention further comprises a pharmaceutical composition (PZ) comprising the amine (AM), or the carbonic acid adduct (KA) as described above, for use in treatment of atypical pneumonia.

Pharmaceutical compositions that also contain the carbonic acid adduct (KA) are described in WO2019/048590.

In principle, the pharmaceutical formulation (PZ) in the context of this invention means a composition comprising the amine (AM) or carbonic acid adduct (KA), which may additionally comprise further auxiliaries or additives suitable for a pharmaceutical-medical use.

Moreover, the pharmaceutical formulation (PZ) may comprise further bases that do not correspond to the amine (AM) and may be different than the base (BA). In principle, the person skilled in the art will be able to select the additives according to the desired end use. In so doing, they will take account of the desired administration form.

The pharmaceutical formulation (PZ) may in principle be in any suitable dosage form. For example, the pharmaceutical formulation (PZ) may be in capsule form, or in the form of a tablet, solution, ointment, cream, gel, paste, poultice or active ingredient-containing patch.

The pharmaceutical formulation (PZ) may in principle be applied in any suitable administration form. The person skilled in the art will select a suitable dosage form in accordance with the intended administration form. For example, the pharmaceutical formulation (PZ) may be administered orally, by inhalation, by injection, as a patch, cutaneously comprising at least dermal application, application to the eye, nasal application, rectal application and vaginal application.

In the formulation of the pharmaceutical formulation (PZ), the person skilled in the art can fundamentally make use of the methods known in the prior art.

Preferably, the temperature of the mixture of the carbonic acid adduct (KA) and the auxiliaries used and any further bases during the formulation of the pharmaceutical formulation (PZ) is less than 60° C., preferably less than 50° C., more preferably 0 to 50° C.

In the formulation of the pharmaceutical formulation (PZ), preferably in ointment form, it is also possible to use dispersing, preferably by means of an ointment mixer. Preference is given here to using a speed of <2000 rpm.

Prior to processing to give the oral dosage forms described hereinafter, such as tablets, capsules or semisolid dosage forms, the carbonic acid adduct (KA) may be triturated alone or in the presence of further auxiliaries or bases to give powder. The person skilled in the art may in principle make use of the known technical means that are suitable for the respective purpose. For trituration steps, for example, it is possible to use mortars or similarly suitable means. In the trituration steps, preference is given to using a technical auxiliary that minimizes mechanical stress on the carbonic acid adduct. The trituration is preferably effected with a mortar.

The powder thus obtained can then, for example, be pressed to tablets or dispensed into customary capsules or mixed with suitable auxiliaries and processed to give semisolid dosage forms.

One embodiment of the pharmaceutical formulation (PZ) relates to a pharmaceutical formulation (PZ) that comprises the carbonic acid adduct (KA) and is administered orally. In this embodiment, the pharmaceutical formulation (PZ) is preferably administered in capsules, more preferably in hard gelatin or cellulose capsules, more preferably in hard gelatin capsules. It is likewise possible to administer the pharmaceutical formulation (PZ) in tablet form in this embodiment.

The pharmaceutical formulation (PZ) in this embodiment preferably comprises at least one auxiliary (H), preferably selected from starch, especially corn starch and/or rice starch, dextran, cellulose esters and SiO₂.

In addition, the pharmaceutical formulation (PZ) in this embodiment may comprise at least one base (BA1) that does not correspond to the amine (AM) and is identical to or different than base (BA). Base (BA1) is preferably selected from NaHCO₃ and KHCO₃, more preferably NaHCO₃.

The general statements made above with regard to the pharmaceutical formulation (PZ) are preferably also applicable to this embodiment, especially also to the formulation of the pharmaceutical formulation (PZ), where technically applicable in respect of this embodiment.

The pharmaceutical formulation (PZ) in this embodiment preferably comprises:
a) 1% to 99% by weight, more preferably 15% to 95% by weight, of the carbonic acid adduct (KA),
b) 0% to 60% by weight, more preferably 3% to 50% by weight, of base (BA1) and 1% to 90% by weight, more preferably 2% to 75% by weight, of auxiliary (H), based on the total weight of the pharmaceutical formulation.

A further embodiment of the pharmaceutical formulation (PZ) relates to a semisolid pharmaceutical formulation (PZ) which comprises the carbonic acid adduct (KA) and is administered cutaneously. The pharmaceutical formulation (PZ) in this embodiment can be applied, for example, in ointment form, or in the form of a cream, gel, paste, poultice or active ingredient-containing patch.

The general statements made above with regard to the pharmaceutical formulation (PZ) are preferably also applicable to this embodiment, especially also to the production of the pharmaceutical formulation (PZ), where technically applicable in respect of this embodiment.

The pharmaceutical formulation (PZ) in this embodiment preferably comprises at least one auxiliary (H1) selected from paraffins, especially viscous and mobile paraffins, wool wax, wool wax alcohols, hydrophobic base gel, vegetable oils, animal fats, synthetic glyceride, liquid polyalkylsiloxanes, waxes, vaseline and starch, especially corn starch, preferably vaseline.

Viscous paraffins (Paraffinum subliquidum) mean paraffins having a viscosity of 110 to 230 mPas, while mobile paraffins (Paraffinum perliquidum) have a viscosity of 25 to 80 mPas.

The pharmaceutical formulation (PZ) in this embodiment preferably comprises:
a) 0.1% to 40% by weight, preferably 0.4% to 10% by weight, of the carbonic acid adduct (KA) and
b) 60% to 99.9% by weight, preferably 80% to 96% by weight, of the auxiliary (H1), based on the total amount of the pharmaceutical formulation (PZ).

A further embodiment of the pharmaceutical formulation (PZ) comprising the carbonic acid adduct (KA) relates to a pharmaceutical formulation which is administered by the parenteral, nasal and/or inhalative route.

The general statements made above with regard to the pharmaceutical formulation (PZ) are preferably also applicable to this embodiment, especially also to the formulation of the pharmaceutical formulation (PZ), where technically applicable in respect of this embodiment.

The pharmaceutical formulation (PZ) in this embodiment is preferably in the form of a solution (A2) comprising the carbonic acid adduct (KA), dissolved CO₂ and at least one auxiliary (H2).

The auxiliary (H2) is preferably selected from an alkali metal halide or alkaline earth metal halide, more preferably NaCl and MgCl₂, even more preferably NaCl. The auxiliary (H2) may be identical to the salt (S). Stated amounts based on the auxiliary (H2), where this is identical to the salt (S) in individual embodiments, in the context of this invention, relate to additional amounts of the auxiliary (H2) that have not been introduced into the pharmaceutical formulation (PZ) in the form of the salt (S) as part of the carbonic acid adduct (KA).

Solution (A2) is preferably obtained by introduction of CO₂ into a solvent. The solvent is preferably water. For production of solution (A2), the CO₂ is preferably introduced into the solvent at a temperature of 0 to 8° C., more preferably at 0 to 5° C. The CO₂ can be introduced into the solvent in the form of the gas or in solid form, for instance as dry ice. The CO₂ is preferably introduced into the solvent in the form of the gas. The CO₂ may also be introduced into the solution under pressure until attainment of the desired concentration, as described above for component step a2) for instance.

For production of solution (A2), the CO₂ is preferably introduced into the solvent up to a concentration of at least 3 g/l, more preferably up to 4 g/l, even more preferably 4 g/I to 8 g/l.

The CO₂ which is used for production of solution (A2) has at least a purity of 99.9%, and more preferably also a quality suitable for pharmaceutical applications, as defined above.

In this embodiment, the pharmaceutical formulation (PZ), if it is obtained by dissolving the carbonic acid adduct (KA) in solution (A2), preferably comprises:
ma) 0.05 to 100 mg/ml, more preferably 0.08 to 50 mg/ml, of the carbonic acid adduct (KA) and
b) 0 to 20 mg/ml, more preferably 3 to 10 mg/ml, of the auxiliary (H2) based in each case on the total volume of the pharmaceutical formulation (PZ).

### EXAMPLES OF THE INVENTION

### 1. Working Example 1, Preparation of the Carbonic Acid Adduct (KA)

### 1.1 Materials:

Amine (AM): 68.8 to 110.1 g of procaine hydrochloride (for example ultrapure, for use as active pharmaceutical ingredient; Ph. Eur. or in a quality suitable for that purpose)
Base (BA): 22.2 to 33.9 g of sodium hydrogencarbonate (for example ultrapure, or in a quality suitable for that purpose),
Solvent: 630 to 900 ml of water (Aqua ad iniectabilia)
CO₂: at least 12.0 g/I carbon dioxide from steel pressurized gas bottles (CO₂ in suitable quality)
Dry ice for provision of cold mixtures and for cooling
Methanol, technical grade for provision of cold mixtures

### 1.2 Step a)

Water (e.g. Aqua ad iniectabilia) is introduced into a cleaned plastic squeeze bottle up to the mark (about 800 to 900 ml) and precooled to 5° C. in a refrigerator (3 to 8° C.) or by external cooling for at least 1 h.

A carbon dioxide-saturated carbonic acid solution is prepared. For this purpose, CO₂ is introduced at intervals into the precooled water under pressure (1.6 to 8 bar). Hissing (escaping gas via the pressure relief valve) indicates saturation of the solution with CO₂. Saturation is monitored via weight, until 4.0 to 6.0 g of CO₂ (corresponding to 4.5 to 7.5 g/l) has dissolved. The saturated solution has a pH of ≤4.3 to 4.8. This carbonated water is sealed immediately and stored in a refrigerator for at least 1 h.

### 1.3 Step b)

An initial charge of 21.2 g of sodium hydrogencarbonate in a second plastic squeeze bottle is admixed with 320 ml of cooled carbonated water, and dissolved by upturning the bottle.

### 1.4 Step c)

The equivalent amount of solid procaine hydrochloride is added to this solution at constant temperature, forming a virtually neutral solution which, after addition of a further 320 ml of cold carbonated water, gives a clear, slightly acidic solution. The solution is enriched with CO₂. The solution thus prepared is stored in a refrigerator for at least 1 h.

Subsequently, the solution is once again conditioned with CO₂ until a CO₂ concentration of 12 g/l in the solution has been attained. The pH is monitored by means of pH indicator sticks. The pH is s 6.6.

### 1.5 Step d)

Round-bottom flasks are precooled. For freezing, the reaction solution is measured out in a precooled measuring cylinder, transferred in portions to round-bottom flasks and frozen by immersion into a dry ice/methanol cold mixture (<−60° C.) by the shell freezing method within 1.5 to 3.5 min for each flask (˜200 rpm). The angle of immersion of the flask in the rotary evaporator is set to about 40°.

### 1.6 Step e)

The flasks containing the material thus frozen are closed with a ground glass stopper and stored intermediately in a freezer at −15 to −20° C. for 2 to 4 days.

### 1.7 Step f)

The flasks that have been subjected to such temperatures are ensheathed with precooled Styropor vessels and immediately connected individually to an evacuated (0.060±0.01 mbar, about −46° C., leak-tightness test) freeze-drying system, via a flexible rubber cone. The valve taps are opened cautiously, and the individual flasks are put under vacuum. All flasks must ultimately be evacuated.

For monitoring of the process, temperature sensors are positioned in the bottom of the Styropor shell, which record the entire temperature profile throughout the drying operation. Before the start of lyophilization, the temperature sensors indicate temperatures of <−5° C. During the lyophilization, the pressure is 0.07±0.02 mbar. This sublimation pressure is attained within 4 h and maintained throughout the lyophilization time. The cooling chamber is adjusted to a temperature of 9 to 15° C. throughout the drying operation. The endpoint of the lyophilization is ascertained by graph from the temperature progression recordings. The total drying time is not more than 52 h. The dry lyophilizate is transferred to a brown glass vessel with a twist-off lid, provided with a desiccant pouch and stored in a refrigerator at 0 to 15° C.

### 2. Examples of Pharmaceutical Formulations (PZ)

### 2.1 Capsules and Tablets

There follows a description by way of example of the composition of the pharmaceutical formulation (PZ) according to the invention in the embodiment as a capsule or tablet for procaine as amine (AM). For the production of the capsules, it is possible to use commercial two-piece capsules in the commercial sizes (5 to 000), which are filled with the powder containing the carbonic acid adduct (KA), comprising procaine as amine (AM) (trituration of the carbonic acid adduct (KA) active ingredient, comprising procaine as amine (AM), optionally with auxiliaries, fillers and flow regulators). The carbonic acid adduct (KA) was produced according to example 1. It has been found that hard gelatin capsules are of better suitability compared to cellulose capsules with regard to stability. For instance, the filled hard gelatin capsules, by way of example for hard gelatin capsules with 60 and 100 mg of active ingredient according to table 1, do not show any changes even after storage in a refrigerator for 12 months and are thus stable. Stability was examined by IR spectroscopy. For instance, in the case of hard gelatin capsules, no procaine was detected by IR spectroscopy within the 12-month period, whereas a procaine band was measured in the IR spectrum after only a few days for cellulose capsules.

The content was determined at room temperature with UV/VIS spectroscopy. For formulation medicaments, according to Pharmacopoea Europaea point 2.9.6, based on the total content including by-products, a tolerance range of ±15% is stipulated.

The current and generally customary pharmaceutical rules for production of (formulation) medicaments are applied (for example Pharmacopoea Europaea, Deutscher Arzneimittel-Codex [German Medicaments Code]).

TABLE 1
Example composition of capsules with added
NaHCO₃ and carbonic acid adduct (KA) as active
ingredient, produced according to working example 1.

| | Amount of active ingredient [mg] | | | |
|---|---|---|---|---|
| | 30 | 60 | 100 | 120 |
| Addition, e.g. NaHCO₃ | 21 | 42 | 71 | 84 |
| Filler, e.g. corn starch | 120 | 90 | 120 | 90 |
| incl. SiO₂ | | | | |
| Capsule size | 1 | 1 | 0 | 0 |

TABLE 2
Example composition of tablet with added
NaHCO₃ and carbonic acid adduct (KA) as active
ingredient, produced according to working example 1.

| | Amount of active ingredient [mg] | | | |
|---|---|---|---|---|
| | 30 | 60 | 100 | 120 |
| Addition, e.g. NaHCO₃ | 21 | 42 | 71 | 84 |
| Filler, e.g. corn starch | 2.5 | 5 | 8.5 | 10 |
| incl. SiO₂ | | | | |

### 2.2 Ointments

There follows an elucidation of the composition of the pharmaceutical formulation (PZ) according to the inention in the embodiment as an ointment, by wasy of example for procaine as amine (AM) in the carbonic acid adduct (KA). In the preparation of the ointment, the current and generally customary pharmaceutical rules for production of (formulation) medicaments are applied (for example Pharmacopoea Europaea, Deutscher Azneimettel-Codex). Relatively high shear forces are avoided in the production of the ointment. Moreover, the temperature in the course of formulation is kept below 60° C. even locally. Thus, the mortar-crushed carbonic acid adduct (KA) comprising procaine as amine (AM) is introduced into the ointment base, for example vaseline, in a standard mortar or a Fanta-type mortar adjusted to a temperature of 40 to 45° C. by means of a water bath. Alternatively, it is also possible to use electrical mixing systems as used customarily in pharmacy.

TABLE 3
Example composition of ointment and carbonic acid adduct (KA)
as active ingredient produced according to working example 1.

| | Content [%] | | | | |
|---|---|---|---|---|---|
| | 0.5 | 1.0 | 1.25 | 2 | 4 |
| Amount of active | 25 | 50 | 62.5 | 100 | 200 |
| ingredient [mg] | | | | | |
| Ointment base [g] | 5 | 5 | 5 | 5 | 5 |
| (e.g. vaseline) | | | | | |

### 2.3 Parenteral Solutions

For the provision of a parenteral solution containing the carbonic acid adduct (KA) comprising procaine as amine (AM), the required amount of water (Aqua ad iniectabilia) is cooled to about 5±3° C. in a suitable vessel with a stirrer bar or the like, and left at that temperature. The water is enriched with gaseous carbon dioxide in the required quality to about 3.2 g/l. The appropriate amount of carbonic acid adduct (KA) comprising procaine as amine (AM) and sodium chloride for an isotonic content is dissolved in this carbonated water.

Alternatively, water at a temperature of about 5±3° C. is enriched with CO₂ under pressure in a closed system such that there is a distinct surplus (4.5 to 7.5 g/I). The corresponding amounts of carbonic acid adduct (KA) comprising procaine as amine (AM) and sodium chloride are likewise added to this carbonated water.

This cold solution provided with the carbonic acid adduct (KA) comprising procaine as amine (AM) and sodium chloride is sterile-filtered under suitable ambient conditions and dispensed into appropriate vials. The current and generally customary pharmaceutical rules for production of (formulation) medicaments are applied (for example Pharmacopoea Europaea).

TABLE 4
Example compositions for parenteral formulations with
added NaCl and carbonic acid adduct (KA) as active
ingredient, produced according to working example 1.

| | Infusion | | | Injection | | |
|---|---|---|---|---|---|---|
| Content [%] | 0.1 | 0.2 | 0.3 | 1 | 2 | 3 |
| Amount of active | 50 | 100 | 150 | 50 | 100 | 150 |
| ingredient [mg] | | | | | | |
| Addition (NaCl) [mg] | 440 | 430 | 425 | 37 | 29 | 25 |
| Total volume [ml] | 50 | 50 | 50 | 5 | 5 | 5 |

### 3. Solubility in Octanol

Solubility tests in octanol were conducted, and the content in the organic phase was examined by UV/VIS spectroscopy, as were the pH values.

TABLE 5
Solubility of various dosage forms

| Substance | Content | pH |
|---|---|---|
| Procaine | completely dissolved (100%) | 8.5 |
| Procaine hydrochloride | 8% | 4.5 |
| (Proc HCl) | | |
| Carbonic acid adduct of | 74% | 7.5 |
| procaine | | |

The pH values ascertained show that the carbonic acid adduct of procaine is converted as such and not into procaine and in this form is soluble in the organic phase and hence membrane-permeable. It is likewise apparent from the measurement of content by UV/VIS spectroscopy that the carbonic acid adduct of procaine, in respect of lipophilicity, is more similar to procaine than procaine HCl. It thus behaves like the basic component—the lipid-soluble base which is formed depending on the pH. The carbonic acid adduct of procaine has this property irrespective of pH, meaning that it need not be converted to lipophilic form by a change in pH like procaine HCl. Procaine HCl has a much lower value, which, at 8%, is in the order of magnitude for protein binding of 6%.

### LITERATURE

1. Clayville L R. 2011. Influenza update: a review of currently available vaccines. P T 36:659-84.
2. Taubenberger J K, Kash J C, Morens D M. 2019. The 1918 influenza pandemic: 100 years of questions answered and unanswered. Sci Transl Med 11.
3. Morens D M, Taubenberger J K, Fauci A S. 2008. Predominant role of bacterial pneumonia as a cause of death in pandemic influenza: implications for pandemic influenza preparedness. J Infect Dis 198:962-70.
4. McCullers J A. 2014. The co-pathogenesis of influenza viruses with bacteria in the lung. Nat Rev Microbiol 12:252-62.
5. Vanderbeke L, Spriet I, Breynaert C, Rijnders B J A, Verweij P E, Wauters J. 2018. Invasive pulmonary aspergillosis complicating severe influenza: epidemiology, diagnosis and treatment. Curr Opin Infect Dis 31:471-480.
6. Nulens E F, Bourgeois M J, Reynders M B. 2017. Post-influenza aspergillosis, do not underestimate influenza B. Infect Drug Resist 10:61-67.
7. Alobaid K, Alfoudri H, Jeragh A. 2020. Influenza-associated pulmonary aspergillosis in a patient on ECMO. Med Mycol Case Rep 27:36-38.
8. Huang L, Zhang N, Huang X, Xiong S, Feng Y, Zhang Y, Li M, Zhan Q. 2019. Invasive pulmonary aspergillosis in patients with influenza infection: A retrospective study and review of the literature. Clin Respir J 13:202-211.
9. Verweij P E, Rijnders B J A, Bruggemann R J M, Azoulay E, Bassetti M, Blot S, Calandra T, Clancy C J, Comely O A, Chiller T, Depuydt P, Giacobbe D R, Janssen N A F, Kullberg B J, Lagrou K, Lass-Florl C, Lewis R E, Liu P W, Lortholary O, Maertens J, Martin-Loeches I, Nguyen M H, Patterson T F, Rogers T R, Schouten J A, Spriet I, Vanderbeke L, Wauters J, van de Veerdonk F L. 2020. Review of influenza-associated pulmonary aspergillosis in ICU patients and proposal for a case definition: an expert opinion. Intensive Care Med 46:1524-1535.
10. Ostrosky-Zeichner L, Casadevall A, Galgiani J N, Odds F C, Rex J H. 2010. An insight into the antifungal pipeline: selected new molecules and beyond. Nat Rev Drug Discov 9:719-27.
11. Tobin J M, Nickolich K L, Ramanan K, Pilewski M J, Lamens K D, Alcorn J F, Robinson K M. 2020. Influenza Suppresses Neutrophil Recruitment to the Lung and Exacerbates Secondary Invasive Pulmonary Aspergillosis. J Immunol 205:480-488.
12. Wiederhold N P, Verweij P E. 2020. Aspergillus fumigatus and pan-azole resistance: who should be concerned?Curr Opin Infect Dis 33:290-297.
13. Duwe S C, Schmidt B, Gartner B C, Timm J, Adams O, Fickenscher H, Schmidtke M. 2021. Prophylaxis and treatment of influenza: options, antiviral susceptibility, and existing recommendations. GMS Infect Dis 9:Doc02.
14. Hurt A C, Besselaar T G, Daniels R S, Ermetal B, Fry A, Gubareva L, Huang W, Lackenby A, Lee R T, Lo J, Maurer-Stroh S, Nguyen H T, Pereyaslov D, Rebelo-de-Andrade H, Siqueira M M, Takashita E, Tashiro M, Tilmanis D, Wang D, Zhang W, Meijer A. 2016. Global update on the susceptibility of human influenza viruses to neuraminidase inhibitors, 2014-2015. Antiviral Res 132:178-85.
15. Ludwig S. 2011. Disruption of virus-host cell interactions and cell signaling pathways as an anti-viral approach against influenza virus infections. Biol Chem 392:837-47.
16. Lampejo T. 2020. Influenza and antiviral resistance: an overview. Eur J Clin Microbiol Infect Dis 39:1201-1208.
17. Haring C, Schroeder J, Löffler B, Engert B, Ehrhardt C. 2021. The local anaesthetic procaine prodrugs ProcCluster<sup>®</sup> and Procaine-hydrochloride impair SARS-CoV-2 replication doi:10.1101/2021.06.07.447335:2021.06.07.447335.
18. Abdelaziz A A, el-Nakeeb M A. 1987. Sporicidal activity of local anaesthetics and their binary combinations with preservatives. J Basic Microbiol 27:403-10.
19. Rodrigues A G, Araujo R, Pina-Vaz C. 2006. Interaction of local anaesthetics with other antifungal agents against pathogenic Aspergillus. Int J Antimicrob Agents 27:339-43.
20. Krammer F, Smith G J D, Fouchier RAM, Peiris M, Kedzierska K, Doherty P C, Palese P, Shaw M L, Treanor J, Webster R G, Garcia-Sastre A. 2018. Influenza. Nat Rev Dis Primers 4:3.
21. Kim H, Webster R G, Webby R J. 2018. Influenza Virus: Dealing with a Drifting and Shifting Pathogen. Viral Immunol 31:174-183.
22. Morens D M, Taubenberger J K. 2018. The Mother of All Pandemics Is 100 Years Old (and Going Strong)!Am J Public Health 108:1449-1454.
23. Taubenberger J K, Reid A H, Lourens R M, Wang R, Jin G, Fanning T G. 2005.

Characterization of the 1918 influenza virus polymerase genes. Nature 437:889-93.
24. Morris D E, Cleary D W, Clarke S C. 2017. Secondary Bacterial Infections Associated with Influenza Pandemics. Front Microbiol 8:1041.
25. Podewils L J, Liedtke L A, McDonald L C, Hageman J C, Strausbaugh L J, Fischer T K, Jernigan D B, Uyeki T M, Kuehnert M J, Infectious Diseases Society of America Emerging Infections N. 2005. A national survey of severe influenza-associated complications among children and adults, 2003-2004. Clin Infect Dis 40:1693-6.
26. Iverson A R, Boyd K L, McAuley J L, Plano L R, Hart M E, McCullers J A. 2011. Influenza virus primes mice for pneumonia from Staphylococcus aureus. J Infect Dis 203:880-8.
27. Ku Y H, Chan K S, Yang C C, Tan C K, Chuang Y C, Yu W L. 2017. Higher mortality of severe influenza patients with probable aspergillosis than those with and without other coinfections. J Formos Med Assoc 116:660-670.
28. Kosmidis C, Denning D W. 2015. The clinical spectrum of pulmonary aspergillosis. Thorax 70:270-7.
29. van de Veerdonk F L, Gresnigt M S, Romani L, Netea M G, Latge J P. 2017. Aspergillus fumigatus morphology and dynamic host interactions. Nat Rev Microbiol 15:661-674.
30. Duan Y, Ou X, Chen Y, Liang B, Ou X. 2020. Severe Influenza With Invasive Pulmonary Aspergillosis in Immunocompetent Hosts: A Retrospective Cohort Study. Front Med (Lausanne) 7:602732.
31. Shah M M, Hsiao E I, Kirsch C M, Gohil A, Narasimhan S, Stevens D A. 2018. Invasive pulmonary aspergillosis and influenza co-infection in immunocompetent hosts: case reports and review of the literature. Diagn Microbiol Infect Dis 91:147-152.
32. Ludwig S. 2009. Targeting cell signalling pathways to fight the flu: towards a paradigm change in anti-influenza therapy. J Antimicrob Chemother 64:1-4.
33. Meineke R, Rimmelzwaan G F, Elbahesh H. 2019. Influenza Virus Infections and Cellular Kinases. Viruses 11.
34. Haasbach E, Muller C, Ehrhardt C, Schreiber A, Pleschka S, Ludwig S, Planz O. 2017. The MEK-inhibitor CI-1040 displays a broad anti-influenza virus activity in vitro and provides a prolonged treatment window compared to standard of care in vivo. Antiviral Res 142:178-184.
35. Holzberg M, Boergeling Y, Schrader T, Ludwig S, Ehrhardt C. 2017. Vemurafenib Limits Influenza A Virus Propagation by Targeting Multiple Signaling Pathways. Front Microbiol 8:2426.
36. Ludwig S, Wolff T, Ehrhardt C, Wurzer W J, Reinhardt J, Planz O, Pleschka S. 2004. MEK inhibition impairs influenza B virus propagation without emergence of resistant variants. FEBS Lett 561:37-43.
37. Marjuki H, Alam M I, Ehrhardt C, Wagner R, Planz O, Klenk H D, Ludwig S, Pleschka S. 2006. Membrane accumulation of influenza A virus hemagglutinin triggers nuclear export of the viral genome via protein kinase Calpha-mediated activation of ERK signaling. J Biol Chem 281:16707-15.
38. Pleschka S, Wolff T, Ehrhardt C, Hobom G, Planz O, Rapp U R, Ludwig S. 2001. Influenza virus propagation is impaired by inhibition of the Raf/MEK/ERK signalling cascade. Nat Cell Biol 3:301-5.
39. Schrader T, Dudek S E, Schreiber A, Ehrhardt C, Planz O, Ludwig S. 2018. The clinically approved MEK inhibitor Trametinib efficiently blocks influenza A virus propagation and cytokine expression. Antiviral Res 157:80-92.
40. Ehrhardt C, Ruckle A, Hrincius E R, Haasbach E, Anhlan D, Ahmann K, Banning C, Reiling S J, Kuhn J, Strobl S, Vitt D, Leban J, Planz O, Ludwig S. 2013. The NF-kappaB inhibitor SC75741 efficiently blocks influenza virus propagation and confers a high barrier for development of viral resistance. Cell Microbiol 15:1198-211.
41. Haasbach E, Reiling S J, Ehrhardt C, Droebner K, Ruckle A, Hrincius E R, Leban J, Strobl S, Vitt D, Ludwig S, Planz O. 2013. The NF-kappaB inhibitor SC75741 protects mice against highly pathogenic avian influenza A virus. Antiviral Res 99:336-44.
42. Mazur I, Wurzer W J, Ehrhardt C, Pleschka S, Puthavathana P, Silberzahn T, Wolff T, Planz O, Ludwig S. 2007. Acetylsalicylic acid (ASA) blocks influenza virus propagation via its NF-kappaB-inhibiting activity. Cell Microbiol 9:1683-94.
43. Deinhardt-Emmer S, Jackel L, Haring C, Bottcher S, Wilden J J, Gluck B, Heller R, Schmidtke M, Koch M, Loffler B, Ludwig S, Ehrhardt C. 2021. Inhibition of Phosphatidylinositol 3-Kinase by Pictilisib Blocks Influenza Virus Propagation in Cells and in Lungs of Infected Mice. Biomolecules 11.
44. Ehrhardt C, Ludwig S. 2009. A new player in a deadly game: influenza viruses and the PI3K/Akt signalling pathway. Cell Microbiol 11:863-71.
45. Ehrhardt C, Marjuki H, Wolff T, Nurnberg B, Planz O, Pleschka S, Ludwig S. 2006. Bivalent role of the phosphatidylinositol-3-kinase (PI3K) during influenza virus infection and host cell defence. Cell Microbiol 8:1336-48.
46. Ehrhardt C, Wolff T, Ludwig S. 2007. Activation of phosphatidylinositol 3-kinase signaling by the nonstructural NS1 protein is not conserved among type A and B influenza viruses. J Virol 81:12097-100.
47. Ehrhardt C, Wolff T, Pleschka S, Planz O, Beermann W, Bode J G, Schmolke M, Ludwig S. 2007. Influenza A virus NS1 protein activates the PI3K/Akt pathway to mediate antiapoptotic signaling responses. J Virol 81:3058-67.
48. Eierhoff T, Hrincius E R, Rescher U, Ludwig S, Ehrhardt C. 2010. The epidermal growth factor receptor (EGFR) promotes uptake of influenza A viruses (IAV) into host cells. PLoS Pathog 6:e1001099.
49. Day A M, Quinn J. 2019. Stress-Activated Protein Kinases in Human Fungal Pathogens. Front Cell Infect Microbiol 9:261.
50. Song J, Pan W, Sun Y, Han J, Shi W, Liao W. 2017. Aspergillus fumigatus-induced early inflammatory response in pulmonary microvascular endothelial cells: Role of p38 MAPK and inhibition by silibinin. Int Immunopharmacol 49:195-202.
51. Denning D W, Hope W W. 2010. Therapy for fungal diseases: opportunities and priorities. Trends Microbiol 18:195-204.
52. Ji Y, Yang F, Ma D, Zhang J, Wan Z, Liu W, Li R. 2012. HOG-MAPK signaling regulates the adaptive responses of Aspergillus fumigatus to thermal stress and other related stress. Mycopathologia 174:273-82.
53. Hagiwara D, Suzuki S, Kamei K, Gonoi T, Kawamoto S. 2014. The role of AtfA and HOG MAPK pathway in stress tolerance in conidia of Aspergillus fumigatus. Fungal Genet Biol 73:138-49.
54. Yang G, Cao X, Ma G, Qin L, Wu Y, Lin J, Ye P, Yuan J, Wang S. 2020. MAPK pathway-related tyrosine phosphatases regulate development, secondary metabolism and pathogenicity in fungus Aspergillus flavus. Environ Microbiol 22:5232-5247.
55. Manfiolli A O, Dos Reis T F, de Assis L J, de Castro P A, Silva L P, Hor J I, Walker L A, Munro C A, Rajendran R, Ramage G, Goldman G H. 2018. Mitogen activated protein kinases (MAPK) and protein phosphatases are involved in Aspergillus fumigatus adhesion and biofilm formation. Cell Surf 1:43-56.
56. Schloer S, Goretzko J, Kuhnl A, Brunotte L, Ludwig S, Rescher U. 2019. The clinically licensed antifungal drug itraconazole inhibits influenza virus in vitro and in vivo. Emerg Microbes Infect 8:80-93.
57. Schloer S, Goretzko J, Pleschka S, Ludwig S, Rescher U. 2020. Combinatory Treatment with Oseltamivir and Itraconazole Targeting Both Virus and Host Factors in Influenza A Virus Infection. Viruses 12.
58. Schloer S, Brunotte L, Mecate-Zambrano A, Zheng S, Tang J, Ludwig S, Rescher U. 2021. Drug synergy of combinatory treatment with remdesivir and the repurposed drugs fluoxetine and itraconazole effectively impairs SARS-CoV-2 infection in vitro. Br J Pharmacol 178:2339-2350.
59. Borgeling Y, Schmolke M, Viemann D, Nordhoff C, Roth J, Ludwig S. 2014. Inhibition of p38 mitogen-activated protein kinase impairs influenza virus-induced primary and secondary host gene responses and protects mice from lethal H5N1 infection. J Biol Chem 289:13-27.
60. Yu J, Sun X, Goie J Y G, Zhang Y. 2020. Regulation of Host Immune Responses against Influenza A Virus Infection by Mitogen-Activated Protein Kinases (MAPKs).

### Microorganisms 8.

61. Scheuch G, Canisius S, Nocker K, Hofmann T, Naumann R, Pleschka S, Ludwig S, Welte T, Planz O. 2018. Targeting intracellular signaling as an antiviral strategy: aerosolized LASAG for the treatment of influenza in hospitalized patients. Emerg Microbes Infect 7:21.

1. An amine (AM) of the general formula (I) 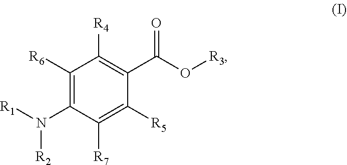
where, in formula (I),
R₁ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₂ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₃ is —(CH₂)ₙNR₈R₉;
n is 1 to 5, preferably 1 to 3, more preferably 1 to 2,
R₃ is (C₁₋₁₀)alkyl, preferably (C₁₋₂)alkyl,
R₉ is (C₁₋₁₀)alkyl, preferably (C₁₋₂)alkyl;
R₄ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or halogen;
R₅ is H, halogen, (C₁₋₁₀)alkyl, (C₂₋₁₀)alkenyl, (C₅-C₁₄)aryl, (C₅-C₁₀)heteroaryl or —O—(C₁₋₁₀)alkyl, preferably H, halogen, (C₁₋₁₀)alkyl or —O(C₁₋₁₀)alkyl, more preferably H or halogen;
R₆ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
R₇ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O—(C₁₋₁₀)alkyl;
where the amine of formula (I) may optionally also be used in the form of a salt,
for use in treatment of infections with Aspergillus fumigatus and of superinfections with influenza viruses and Aspergillus spp.

2. A carbonic acid adduct (KA) comprising at least one structural element of the general formula (II), (III) and/or (IV) 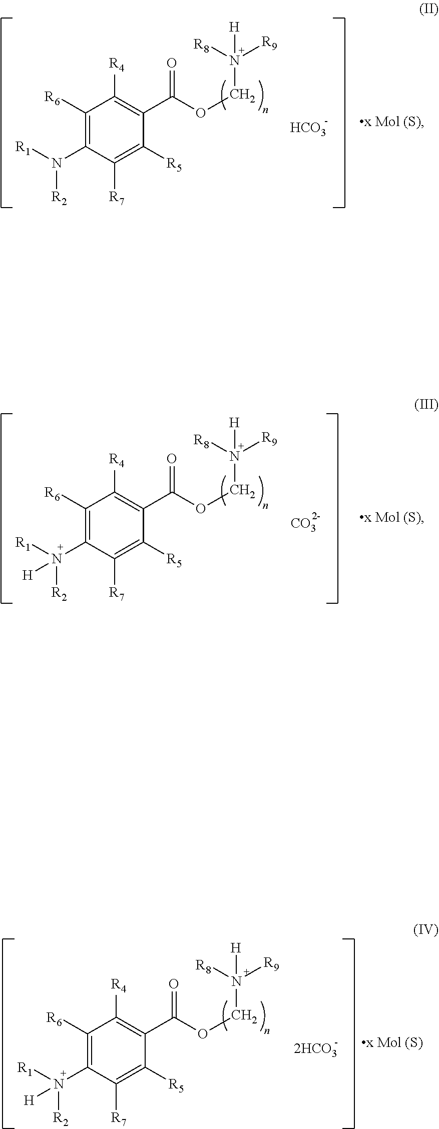
where, in formulae (II), (III), and (IV),
R₁ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₂ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₃ is —(CH₂)ₙNR₈R₉;
n is 1 to 5, preferably 1 to 3, more preferably 1 to 2,
R₃ is (C₁₋₁₀)alkyl, preferably (C₁₋₂)alkyl,
R₉ is (C₁₋₁₀)alkyl, preferably (C₁₋₂)alkyl;
R₄ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or halogen;
R₅ is H, halogen, (C₁₋₁₀)alkyl, (C₂₋₁₀)alkenyl, (C₅-C₁₄)aryl, (C₅-C₁₀)heteroaryl or —O—(C₁₋₁₀)alkyl, preferably H, halogen, (C₁₋₁₀)alkyl or —O(C₁₋₁₀)alkyl, more preferably H or halogen;
R₆ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
R₇ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
x is 0.5 to 30;
(S) is a salt,
for use in treatment of infections with Aspergillus fumigatus and of superinfections with influenza viruses and Aspergillus spp.

3. A carbonic acid adduct (KA) comprising carbonic acid, at least one amine (AM) of the general formula (I) and at least one salt (S), 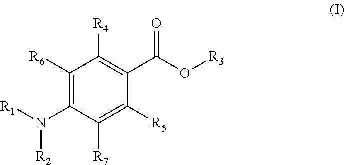
where, in formula (I),
R₁ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₂ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₃ is —(CH₂)ₙNR₈R₉;
n is 1 to 5, preferably 1 to 3, more preferably 1 to 2,
R₃ is (C₁₋₁₀)alkyl, preferably (C₁₋₂)alkyl,
R₉ is (C₁₋₁₀)alkyl, preferably (C₁₋₂)alkyl;
R₄ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or halogen;
R₅ is H, halogen, (C₁₋₁₀)alkyl, (C₂₋₁₀)alkenyl, (C₅-C₁₄)aryl, (C₅-C₁₀)heteroaryl or —O—(C₁₋₁₀)alkyl, preferably H, halogen, (C₁₋₁₀)alkyl or —O(C₁₋₁₀)alkyl, more preferably H or halogen;
R₆ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
R₇ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
where the at least one amine of formula (I) may optionally also be used in the form of a salt;
preparable by a process comprising the steps of:
a) providing a solution (A) comprising at least one solvent, and CO₂ dissolved in the at least one solvent,
optionally b) dissolving a base (BA) that does not correspond to the amine (AM) in solution (A) to obtain solution (A1),
c) dissolving the at least one amine (AM) in solution (A) or (A1) to obtain solution (B),
d) freezing the solution obtained on conclusion of step c),
e) storing the solution frozen in step d) at −100 to 0° C. for no longer than 4 days;
for use in treatment of infections with Aspergillus fumigatus and of superinfections with influenza viruses and Aspergillus spp.

4. A pharmaceutical composition (PZ) comprising the amine (AM) as claimed in claim 1, or the carbonic acid adduct as claimed in either of claims 2 and 3 for use in treatment of infections with Aspergillus fumigatus and of superinfections with influenza viruses and Aspergillus spp.

5. The amine for use as claimed in claim 1, the carbonic acid adduct (KA) for use as claimed in either of claims 2 and 3, and the pharmaceutical composition (PZ) for use as claimed in claim 4, wherein
i) the amine of the general formula (I) and in the general formulae (II), (III) and (IV) is selected from the group consisting of 2-(N,N-diethylamino)ethyl 4-aminobenzoate (procaine), ethyl 4-aminobenzoate (benzocaine), 2-(diethylamino)ethyl 4-amino-2-chlorobenzoate (chloroprocaine), 2-diethylaminoethyl 4-amino-3-butoxybenzoate (oxybuprocaine), 2-(dimethylamino)ethyl 4-(butylamino)benzoate (tetracaine), preferably 2-(N,N-diethylamino)ethyl 4-aminobenzoate (procaine), and/or
ii) the salt (S) is a salt composed of at least one cation selected from Na⁺, K⁺, Li⁺, Mg²⁺, Zn²⁺, Fe²⁺, Fe³⁺ and Mn²⁺, preferably Na⁺, and at least one anion selected from Cl⁻, Br⁻, I⁻, F⁻, SO₄²⁻, SO₃²⁻, HSO₄⁻, HSO₃⁻, —HCO₃⁻, CO₃²⁻, PO₄³⁻, HPO₄²⁻, H₂PO₄⁻, SiO₄⁴⁻, AlO₂⁻, SiO₃⁻ and/or [AlO₂)₁₂(SiO₂)₂]²⁻, preferably Cl⁻ and Br⁻, more preferably Cl⁻.

6. The carbonic acid adduct (KA) for use as claimed in claims 3 and 5, wherein step a) comprises at least one of the following component steps:
a1) cooling the solvent, preferably water, to 3 to 8° C., preferably 5° C. and/or
a2) introducing CO₂ into the solvent, preferably up to a saturation concentration of 3 to 10 g/l, more preferably up to a saturation concentration of 4.5 to 7.5 g/l, where the pH of the solution after the saturation with CO₂ is preferably ≤3.0 to 6.0, even more preferably ≤4.3 to 4.8, and/or
a3) storing solution (A) at 1 to 10° C., preferably for at least 30 min, more preferably for at least 50 min, even more preferably for at least 60 min; up to at most 5 days (120 h); storage is preferably effected at 3 to 8° C. preferably for at least 30 min, more preferably for at least 50 min, even more preferably for at least 60 min; up to at most 5 days (120 h);
step a) preferably comprises all component steps a1), a2) and a3);
component steps a1), a2) and a3) are preferably performed in the sequence of a2) after a1), and a3) after a2).

7. The carbonic acid adduct (KA) for use as claimed in any of claims 3, 5 and 6, wherein
i) the base (BA) in step b) is a hydrogencarbonate or a carbonate, more preferably a hydrogencarbonate, even more preferably sodium hydrogencarbonate, and/or
ii) the content of CO₂ in the solution which is subjected to step d) is at least 6 g/l, preferably at least 10 g/l, more preferably at least 12 g/l, even more preferably at least 14 g/I and most preferably at least 15 g/l, and the amine (AM) may also be used in the form of a salt.

8. The carbonic acid adduct (KA) for use as claimed in any of claims 3 and 5 to 7, wherein step c) comprises at least one of the following component steps:
c1) dissolving the at least one amine (AM) in solution (A) or (A1) to obtain solution (B) and/or
c2) adding solution (A) to solution (B) to obtain solution (1) and/or
c3) enriching solution (B) or (1) with CO₂ and/or
c4) storing solution (B) or (1) at 1 to 10° C., preferably 3 to 8° C., for at least 1 h, preferably 24 h to 120 h, even more preferably 24 to 72 h, and/or
c5) enriching solution (B) or (1) with CO₂ to a concentration of at least 6 g/1, preferably at least 10 g/1, more preferably at least 12 g/1, even more preferably at least 14 g/I and most preferably at least 15 g/l;
where, optionally,
i) the concentration of the amine (AM) in solution (B) or, in the case of execution of component step c2), in solution (1), is 0.01 to 0.25 g/ml, preferably 0.03 to 0.20 g/ml, more preferably 0.08 to 0.15 g/ml, and/or
ii) the pH of solution (B) or (1) after performance of step c5) is s 7.0 and/or
iii) the ratio of the amine (AM) to the base (BA), in the case of performance of step b), in solution (B) is 2 to 5, more preferably 3 to 4, even more preferably 3.23 to 3.26 [g/g], and/or
iv) in step c1), the at least one amine (AM) comprises the at least one amine (AM) as acid addition salt, preferably as hydrohalide, hydrogensulfate, hydrogensulfite, hydrogenphosphate, hydromesylate, hydrotosylate, hydroacetate, hydroformate, hydropropanoate, hydromalonate, hydrosuccinate, hydrofumarate, hydroxalate, hydrotartrate, hydrocitrate, hydromaleate, more preferably as hydrochloride or hydrobromide;
step c) preferably comprises all component steps c1), c2), c3), c4) and c5);
component steps c1), c2), c3), c4) and c5) are preferably performed in the sequence of c2) after c1), c3) after c2), c4) after c3), c5) after c4).

9. The carbonic acid adduct (KA) for use as claimed in any of claims 3 and 5 to 8, wherein, in step d):
i) solution (B) or (1) is frozen at −100° C. to −20° C., preferably at −90° C. to −30° C., even more preferably at −80 to −40° C. and most preferably at −70 to −50° C., and/or
ii) solution (B) or (1) is frozen within 0.3 to 60 min, preferably within 1 to 30 min, more preferably within 1.1 to 10 min, even more preferably within 1.5 to 5 min, and/or
iii) the vessel in which solution (B) or (1) is present during the freezing operation is rotated, preferably in cooling medium, at 10 to 1000 rpm, preferably at 50 to 600 rpm, more preferably at 100 to 400 rpm and even more preferably at 200 to 300 rpm, and/or
iv) solution (B) or (1) is frozen at a cooling rate of 10 to 100 K/min, preferably at 20 to 80 K/min, more preferably at 30 to 70 K/min and especially preferably at 40 to 60 K/min.

10. The carbonic acid adduct (KA) for use as claimed in any of claims 3 and 5 to 8, wherein, in step e):
i) the frozen solution (B) or (1) is stored for 1.5 to 4 days, preferably for 2.5 to 4 days, and/or
ii) the frozen solution (B) or (1) is preferably stored at −50 to 0° C., more preferably at −30 to −5° C., even more preferably at −25 to −10° C., especially preferably at −20 to −15° C.

11. The carbonic acid adduct (KA) for use as claimed in any of claims 3 and 5 to 10, the method comprises a further step f) which is performed after step e):
f) drying the solution stored in step e) to obtain dried carbonic acid adduct (KA), wherein, in step f), optionally,
i) the water is removed from solution (B) or (1) down to a residual content of <0.8% by weight, preferably <0.1% by weight, based on the total weight of the drying product (C), and/or
ii) CO₂ not bound within the carbonic acid adduct (KA) is removed from solution (B) or (1) down to a residual content of <0.8% by weight, preferably <0.1% by weight, based on the total weight of the drying product (C), and/or
iii) the drying is conducted by lyophilization and/or
iv) during the drying, the pressure is 0.01 to 30 mbar, preferably 0.02 to 20 mbar, more preferably 0.03 to 10 mbar, even more preferably 0.03 to 0.5 mbar and most preferably 0.05 to 0.1 mbar, and is preferably maintained throughout the drying operation, and/or
v) the pressure during the drying in iv) is attained within 10 h, preferably within 7 h, more preferably within 5 h and especially preferably within 4 h from commencement of evacuation, and/or
vi) the temperature throughout the drying in step f) is 0 to 20° C., preferably 4 to 18° C., more preferably 8 to 16° C., and/or
vii) the total drying time is 10 to 60, preferably 30 to 55 h, more preferably 41 to 52 h.

12. The amine (AM) as claimed in claim 1, and the carbonic acid adduct (KA) for use as claimed in any of claims 2, 3 and 5 to 11 and the pharmaceutical composition (PZ) for use as claimed in claim 4, in treatment of superinfections with influenza viruses and Aspergillus spp., preferably Aspergillus fumigatus.

13. The amine (AM) as claimed in claim 1, and the carbonic acid adduct (KA) for use as claimed in any of claims 2, 3 and 5 to 12 and the pharmaceutical composition (PZ) for use as claimed in claim 4 and 12, wherein administration is by the oral, parenteral, nasal, inhalative or cutaneous route.
